(19) United States
(12) Patent Application Publication
(10) Pub. No.: US 2025/0236831 A1
(43) Pub. Date: Jul. 24, 2025

(54) VESSEL FOR USE IN PROCESSES ASSOCIATED WITH A LIQUID SOLUTION OF FERMENTABLE SUGARS, A METHOD OF USING THE VESSEL, AND AN ASSOCIATED CLOSED HYDRAULIC SYSTEM

(71) Applicant: ZEPTO PROCESS TECHNOLOGIES LTD, Pyrford, Surrey (GB)

(72) Inventor: Peter Mackie, Pyrford, Surrey (GB)

(21) Appl. No.: 18/855,863

(22) PCT Filed: Mar. 31, 2023
(86) PCT No.: PCT/GB2023/050849

§ 371 Date: Oct. 10, 2024

(30) Foreign Application Priority Data
Apr. 11, 2022 (GB) 2205285.6
Aug. 12, 2022 (GB) 2211827.7

(51) Int. Cl. C12M 1/00; C12M 1/02

(52) U.S. Cl. CPC C12M 29/14; C12M 23/34; C12M 23/40; C12M 41/18

(57) ABSTRACT

In one aspect, a vessel is provided for use in processes associated with a liquid solution of fermentable sugars, the vessel having opposing first and second ends. The vessel further including: a first tube having one or more holed side walls; a second tube having at least one holed side wall, wherein the first tube is located inside the second tube; and a container, wherein the first tube is connected to the container at an open end thereof via a first end piece, and wherein the second tube is located inside and connected to the container. Liquid can flow through the one or more holed side walls of the first tube and the at least one holed side walls of the second tube, thereby allowing the liquid to continuously flow through the volume defined by the first and second tubes. Control of temperature and pH inside the volume is improved.

### FIELD OF DISCLOSURE

The present invention relates to a vessel for processes associated with a liquid solution of fermentable sugars, a method of using the vessel, and an associated closed hydraulic system. The invention is particularly relevant to the production of grain based liquid solutions of fermentable sugars, particularly for use in alcoholic beverages, including beer brewing and grain based spirit production such as whiskey and gin, and/or the processing and fermentation thereof. Alternatively, the present invention may relate to a vessel for hot liquor enzymatic cleaning, a method of using the vessel for enzymatic cleaning and an associated closed hydraulic system.

### BACKGROUND

Liquid solutions of fermentable sugars (also called ‘wort’) can be produced using a mashing process. After a production process, liquid solutions can undergo additional processes, such as lautering processes, boiling processes and fermentation processes in order to adjust the characteristics (e.g. sugar content, taste etc.) of the liquid solution. In an example, producing grain based alcoholic liquids includes a mashing process, a lautering process that can include a sparging process, a boiling process and a fermentation process. In the mashing process, grain is steeped in a liquid to produce wort. The wort is then separated from the grain in a lautering process. The wort is then boiled together with additives to provide flavour to the final product. The boiled wort is then cooled, and combined with yeast in the fermentation process. In beer brewing, the grain is usually barley, but other grains, such a wheat or oats, can be used, and the additives during the boiling process include hops. Distillation of spirits typically uses grains such as barley, corn, maize, rye and wheat. A single grain can be used, or a combination of grains can be used. For example, the grain for Scotch whisky may be predominantly barley in combination with other grains (e.g. maize or wheat). Grain is initially malted, and the chaff (and any other foreign matter) removed. The grain can be crushed (to varying degrees depending on recipe and/or apparatus) to create grist before being put through the mashing process to create a liquid solution of fermentable sugars (also termed ‘wort’).

Mashing is at the core of the brewing or spirit production process. Grist is placed in a suitable vessel, to form a grain bed in that vessel. The grist is hydrated and heated inside the vessel, for example, as a mash tun, to gelatinize its starches and activate the enzymes that lead to the conversion of starches into fermentable sugars. The key efficiency metric of the mash process is the percentage of fermentable sugars that are extracted, as it is these sugars that are ultimately converted into alcohol in the fermentation process.

The gelatinization and enzymatic activity both require free water. For example, enzymes, known as amylases, can break down starch into constituent sugars by hydrolysis, in which a molecule of water assists enzymes in breaking chemical bonds in the starch to release simple sugar molecules. The liquid used to hydrate the grist is call ‘liquor’ (sometimes called ‘brewing liquor’ when brewing), and can be just water or can be water plus other additives (such as minerals). For example, different minerals in the liquor can adjust the pH, which in turn can affect the activation of different enzyme groups during a mashing process. This can improve the yield of fermentable sugars extracted by those enzyme groups. The mixture of grist and liquor is called ‘mash’.

The efficiency of sugar extraction from grist is dependent on many factors, such as temperature, pH, time and mash thickness—see http://braukaiser.com/wiki/index.php/Understanding_Efficiency. As different enzymes in the grist activate in different temperature and pH ranges, control of the temperature and pH of the liquor-grist mixture is important. The pH and temperature of the liquor can be controlled when creating the mash. Heating the mash can be done in a number of ways, including heating the mash tun separately from the outside and/or a extracting a portion of the mash from the mashing tun, heating that portion of the mash separately and reintroducing that heated mash into the mashing tun. In industrial breweries, a small number of discrete temperature steps are used to maximise the activity of some of the enzyme groups (e.g. in a decoction mashing process, in which a portion of liquid is removed from a mashing vessel and super heated before being replaced). Such a discrete, step-wise temperature increase, where the temperature is relatively quickly raised to a temperature level and held for a predetermined time period before being relatively quickly raised to the next temperature level and held for a predetermined period of time and so forth can be sub-optimal. The temperature levels may be at the outer limits of the activation range for some enzymes, or may miss some of the activation range of some enzymes altogether, thereby reducing the yield of fermentable sugar from the grist used in the mashing process.

In industrial scale brewing, hot liquor and grist are mixed and fed into the mash tun under ambient pressure, sometimes using inert gases to eliminate the risk of Hot-Side Aeration (HSA) during the early stages of mashing. Hot-Side Aeration generally relates to the introduction of oxygen into hot wort. The oxygen can combine with various elements to produce undesirable compounds, which can affect the final product. HSA is typically considered to occur at above 26 degrees Celsius (80 degrees Fahrenheit).

As noted above, the mash thickness (ratio of liquor to grist) has an effect on the efficiency of the mashing process. In part, this relates to the ratio of grist to water in that water is required to extract sugars from starch, however other factors are also relevant. For example, as sugar is released from the grist, the viscosity of the liquor increases. As sugar isn't released at the same rate from all parts of the grist, the liquor will have a higher sugar content in some areas than in others. As well, the more viscous liquor is denser and hence will fall to the bottom of the mashing vessel (mash tun). A local sugar content of the liquor may therefore be high enough to restrict enzymatic activity. The mash tuns typically include mechanical stirrers to ensure a homogenous mix of liquor and grist. The mechanical stirrers assist in mixing the liquor and grist such that the local sugar content in the liquor is evenly distributed. This assists with coating as much of the surface area of the grist with liquor as possible and ensures a more uniform temperature, pH and free water distribution across the liquor, thereby improving the efficiency of the mashing process. Some mash tuns are heated from the outside, for example using steam on the side wall, whereas other conventional heating methods include removing and heating a portion of the grain, before replacing that heated portion of the grain. Mechanical stirrers are used to ensure an even distribution of heat amongst the mixture of liquor and grist.

Wort (including the fermentable sugars) is separated from the grist in a lautering process. For example, the lautering process may include a sparging process, in which the fermentable sugars are rinsed from the grain (grist). During the sparging process, wort is drained from the bottom of the mash tun (or from a separate lautering tun if the mash has been transferred to said lautering tun prior to the sparging process). Sparging liquor (also called sparge water) can be sprayed onto the mash to rinse the grain of the fermentable sugars. In industrial brewing, the sparging liquor is sprayed onto the grain bed with a mechanical sparging arm to wash as much of the fermentable sugar off the grist as possible. To improve the efficiency of the sparging process, the depth of the grain bed is important. If the grain bed is too thin, the grist will not sufficiently settle and filter the sparging liquor. If the grain bed is too thick, the grist can prevent the liquor from flowing through altogether, resulting in a clogged vessel in which the sparging is taking place. This is known as a stuck sparge (e.g. https://www.homebrewersassociation.org/how-to-brew/fly-sparing-vs-batch-sparging/). Accordingly, sparging the mash can involve placing a portion of the mash in a lautering tun at a time to avoid making the grain bed too thick. This slows the lautering process, or requires additional equipment (such as additional lautering tuns, or larger lautering tuns). In smaller scale alcohol production, sparging may be omitted for convenience (i.e. the wort drained from the mashing vessel is the only collection of fermentable sugars). Omitting the sparging process reduces the yield of fermentable sugars from the grist.

Another method of improving the yield of a lautering process is mash press filtration (https://www.brewmation.com/brewing/mash-press). The mash is placed in a mash press, and wort filtered from the grist. The mash can be compacted or pressed in the mash press to extract as much of the liquid as possible.

The processes of mechanical stirring, decoction mashing, lautering and grain pressing deliver high efficiency but at a capital cost that dictates a need for large scale plants and thus longer transport distances to distribute final product to the end consumer with the associated environmental impact.

At the end of the lautering process (which may include a sparging process), the usable sugars will have been collected, and the remaining grist is termed ‘spent grain’. The spent grain must then be removed from the mash tun (or the lautering tun, if the mash was transferred after being formed in the mash tun). This is a cumbersome task, requiring mechanical equipment to move the grain and spray the inside of the mash and lautering tuns clean.

Following the lautering process, the wort is boiled. Boiling sterilizes the wort and also stops any remaining enzymatic processes. To produce a particular flavour, the wort may be boiled with additives. For example, when brewing beer, the wort is boiled with hops. The volume of the wort is also reduced during the boiling process (i.e. the wort becomes more concentrated).

After the boiling process, the wort is cooled while removing the hops. When the wort is at a desired temperature, yeast is added to the wort and a fermentation process takes place. Heat is generated as the yeast converts the sugars in the wort to alcohol and, as such, the heat of the wort during the fermentation process must be monitored and regulated.

Alternatively, the wort may be used for the production of non-alcoholic products, such as grain based syrups, sugars or extracts. Instead of undergoing a fermenting process, the wort may instead be boiled down and concentrated into a thicker solution, or evaporated into solids. Other treatments may also be used, such as clarifying, decolourising, additional filtering or further additions of enzymes to reach the desired ratio of sugars (e.g. http://www.madehow.com/Volume-4/Corn-Syrup.html; https://www.homebrewersassociation.org/how-to-brew/how-malt-extract-is-made/; and https://byo.com/article/brewing-with-corn/).

Exemplarily, malt syrup and malt extract are used in a variety of foods and drinks, for example as a sweetener or binding ingredient. Malt syrup and malt extract are also used in baked goods, including bagels, breads, cookies and cakes; as well as in glazes for meats and in granola. Wort obtained from other grains may also be used to create syrups or extracts.

In a further example, during production of alcoholic liquids, a portion of wort may be removed before the boiling process and boiled down separately into a syrup or extract, before being added back to the main liquid during the boiling. By boiling it down separately, the portion of wort may be more concentrated, and help provide a stronger flavour. Alternatively, externally sourced grain based syrups, sugars, and extracts may be added during production of alcoholic liquids (https://www.homebrewtalk.com/threads/wort-boil-down-technique.567957/).Grain based syrups, sugars and extracts may also be used as an alternative basis for the production of alcoholic liquid without requiring a grain mashing process. For example, those producing alcoholic liquids at home may prefer using pre-produced syrups or extracts, such as malt extract, as a time and space saving means (https://www.brewuk.co.uk/blog/home_brew_beer_using_extract/).

US2017/0283751A1 relates to an apparatus for producing a wort including an outer cylindrical container a lower plate received therein; an inner cylindrical container received in the outer cylindrical container such that a wort circulation space is defined the outer cylindrical container and the inner cylindrical container, wherein a malt screener is received in the inner container above the bottom of the inner cylindrical container to be spaced from the bottom of the inner cylindrical container, wherein the side wall of the inner cylindrical container has a plurality of side holes defined therein, and a wort space is defined between the lower plate and the malt screener in the inner cylindrical container; a wort circulator liquid-communicating with the wort circulation space and the wort space to enable circulation of the wort between the wort circulation space and the wort space; and a heater disposed in the wort circulation space to heat the wort therein. A further inner cylinder can be placed inside the inner cylindrical container and, malt is placed inside a further inner cylinder. Water is then introduced into the system, and steeps the malt under ambient pressure. Both the inner cylindrical container and the further inner cylinder filter water before it reaches the malt. Water then leaves the inner cylindrical container through a malt screener at the bottom of the inner cylindrical container.

DE202015007481U1 relates to a boiler for brewing beer by steeping malt under ambient pressure. The boiler comprises a container through which liquid can flow and which can be filled with malt, which can be removably inserted into the boiler and is provided with a pipeline which has a hydraulic pump and which with its one end opens into the container from below and the other end into the boiler, the hydraulic pump pumping the liquid in the malt container from the one in the boiler or pumps the malt container from the opening end of the pipeline to the other end, characterized in that the malt container has an unperforated wall and an unperforated cover and a perforated bottom, and in that the malt container has a perforated inlet column in its interior, which is detachably connected centrally in the bottom of the malt tank to the pipe end of the end of the pipeline opening into the malt tank, such that a liquid circuit can flow axially from the top of the interior of the malt container to the perforated bottom can be produced.

It is desirable to address issues noted above and to provide better control over the conditions inside a vessel for use in processes associated with a liquid solution of fermentable sugars, while improving the efficiency of such processes.

### Means for Solving the Problem

In accordance with the present invention, there is provided a vessel as set out in claim 1, a method as set out in claim 14 and a closed hydraulic system as set out in claim 17. Other aspects of the invention are set out in the dependent claims.

To address problems in the prior art, a vessel for use in processes associated with a liquid solution of fermentable sugars, the vessel having a first end and a second end, the second end being opposite the first end, is provided. The vessel comprises a first tube having an open end, a closed end and one or more holed side walls that extend from the open end to the closed end; a second tube having at least one holed side wall extending from a connected end of the second tube to a closed end of the second tube, wherein the first tube is located inside the second tube with a volume being defined between the first tube and the second tube; and a container having a first end piece at the first end of the vessel and a second end piece at the second end of the vessel, wherein the first tube is connected to the container at the open end via the first end piece, and wherein the second tube is located inside and connected to the container at the connected end of the second tube. Preferably, the vessel is for use in alcoholic liquid production.

Advantageously, when in use, grist (or hops or yeast) can be placed in a volume defined by the first and second tubes to form a grain bed. Liquid can exit the first tube through holes in the at least one side wall, move through the grain bed under hydraulic pressure, and exit the second tube through holes in the at least one side wall of the second tube. The liquid can therefore continuously pass through the grain bed (e.g. under hydraulic pressure). As the liquid can be continuously moving, it is less likely that areas of high viscosity (i.e. areas of high local sugar content) are formed. Therefore free water is more evenly distributed throughout the grain bed, thereby improving control of the conditions in the vessel and allowing enzymatic conversion to occur across the grain bed in a more uniform manner to improve efficiency of conversion of starch into fermentable sugars. In addition, being able to continuously circulate liquid through the vessel allows the liquid to reach as much of the surface area of the grist as possible without the need for mechanical stirrers. Also, the distance that the liquid has to travel through the grain bed is limited to the distance between the first tube and the second tube. As the liquid travels a shorter distance through the grain bed than a conventional vessel of similar size, the pressure drop between a point at which liquid enters the vessel and a point at which liquid leaves the vessel is reduced (i.e. there is less resistance to the liquid passing through the vessel). In turn, this means the temperature of the liquid is similar across all of the grain bed, which allows better control over the temperature in the vessel. This then allows the temperature to be within desired temperature ranges that correlate to the activation temperature windows of various enzymes in the grist for longer so that a higher proportion of fermentable sugars can be released from a given amount of grist.

In some aspects, the first tube is coaxial with the second tube. The liquid therefore travels the same distance through the grain bed whichever direction it leaves the first tube. As such, the temperature is more easily regulated.

In some aspects, the second tube is coaxial with the container. In use, liquid exiting the second tube into the container is at approximately the same pressure on all sides of the second tube. Temperature and PH levels of liquid in the second tube are therefore more uniform.

In some aspects, the connected end of the second tube comprises a detachable cap that can be detached from the one or more side wall. The second tube can therefore be refilled with grist (or hops or yeast) easily. Preferably, the cap attaches to the at least one holed side wall of the second tube by friction. This allows the cap to move while being attached to the least one holed side walls of the second tube, thereby allowing expansion of a volume defined by the second tube and the first tube (also termed the ‘second volume’ herein) as the grain bed expands, as may happen when liquid is introduced into the vessel.

In some aspects, the first tube is connected to a first pipe connector at the first end of the vessel, the first pipe connector being adapted to connect to a first pipe and to create a liquid path between the first pipe and the first tube.

In some aspects, holes in the first tube are 1 mm or less than 1 mm in at least one dimension. Liquid can therefore flow out of the first tube as a jet to stir the grist. At the same time, the grist is prevented from passing through the holes.

In some aspects, the container further comprises a first subsection, a second subsection and a subsection connector, wherein the first subsection and the second subsection are detachably connected by the subsection connector. The container can therefore be opened and the second tube can be accessed for maintenance and/or filling with grist (or hops or yeast).

In some aspects, the first end piece and/or the second end piece include a frustoconical portion.

In some aspects, the vessel comprises a first holed disc connected to the first end piece and the first tube is connected to the first end piece by the first holed disc. Liquid pushes the grist away from the first end piece of the first tube. In use, grist is therefore agitated in both a vertical and horizontal direction. This assists in keeping the grist at approximately the same temperature, and further reduces clumping.

In some aspects, the vessel comprises a third tube having a holed side wall, wherein the third tube is connected to the second end piece and extends toward the first end. The third tube can filter liquid exiting the vessel, to remove any particulates that may have exited the second tube. In arrangements where the vessel comprises the third tube and the second tube comprises a detachable cap that attaches to the at least one holed side wall of the second tube by friction, the third tube can limit the distance that the detachable cap can move, thereby stopping the cap detaching from the at least one holed side wall of the second tube.

In some aspects, the vessel comprises a second holed disc connected to the second end piece and the third tube is connected to the second end piece by the second holed disc; or the vessel comprises a second holed disc connected to an outlet provided at a side wall of the container. The second holed disc can filter liquid exiting the vessel, to remove any particulates that may have exited the second tube.

In some aspects, the vessel comprises a first holed disc connected to the second end piece. Liquid pushes the grist away from the first end piece of the first tube. In use, grist is therefore agitated in both a vertical and horizontal direction. This assists in keeping the grist at approximately the same temperature, and further reduces clumping.

In some aspects, the vessel comprises a third tube having one or more holed side walls, wherein the third tube is connected to an outlet provided at a side wall of the container. The third tube can filter liquid exiting the vessel, to remove any particulates that may have exited the second tube.

In some aspects, the vessel is: a mashing vessel, operable to hold grain within a volume between the first and second tube. In some aspects, the vessel is a mashing and sparging vessel, operable to hold grain within a volume between the first and second tube. Advantageously, liquid can continuously pass through the vessel, with the holes in the one or more holed side walls of the first tube cause the liquid to spray into the second volume, thereby obviating the need for a separate sparging arm. Further, as sparging can occur in the same vessel as the mashing process, the need for a separate lautering tun and/or mash press is removed without substantially reducing the yield of fermentable sugars. In some aspects, the vessel is a boiling vessel, operable to hold hops within a volume between the first and second tube. In some aspects, the vessel is a fermentation vessel, operable to hold yeast within a volume between the first and second tube. Being able to continuously pump liquid through the vessel allows the conditions of the liquid (e.g. temperature and pH) to be controlled external to the vessel before it is reintroduced to the vessel. This allows greater control over the conditions of the liquid inside the vessel during the boiling or fermentation process.

Some embodiments include a method of using the vessel as discussed above, wherein a first volume is defined by an interior of the first tube, the volume defined between the first tube and the second tube is a second volume, and a third volume is defined between the second tube and the container, the method comprising: feeding liquid into the second volume via the first volume through the one or more holed side wall of the first tube; and directing liquid from the second volume into the third volume through the at least one holed side wall of the second tube. Advantageously, liquid exits the first tube through holes in the at least one side wall, moves through the grain bed under hydraulic pressure, and exits the second tube through holes in the at least one side wall of the second tube. The liquid therefore continuously passes through grist (or hops or yeast) in the second volume (e.g. under hydraulic pressure), thereby allowing the liquid to reach as much of the surface area of the grist as possible without the need for mechanical stirrers.

In some aspects, the method comprises determining that the pH of the liquid is outside of a predetermined range, and adjusting the pH of the liquid. The amount of fermentable sugars released from a given amount of grist can therefore be increased. In some aspects, adjusting the pH of the liquid includes adding salts or minerals to the liquid.

Some embodiments include a closed hydraulic system comprising: a first vessel, wherein the first vessel is a vessel as described above; a pump; a liquid tank; a heat exchanger; a first pipe between the liquid tank and the first vessel; a second pipe between the first vessel and the heat exchanger; and a third pipe between the heat exchanger and the liquid tank, wherein the pump is capable of continuous circulating flow of liquid through the vessel via the first, second and third pipe. Advantageously, liquid in the closed hydraulic system can be continuously pumped around the system. In contrast to conventional systems that steep the grist in liquid, continuously pumping all of the liquid around the system minimises build-up of areas of high viscosity (i.e. there is less chance of areas with higher local sugar content forming), as well as allowing the liquid to be used to agitate the grist. The efficiency of the conversion of starch to fermentable sugars in the system is thereby improved. Additionally, liquid in the closed hydraulic system can be heated by the heat exchanger while it is continuously pumped around the system, and before it is re-introduced into the vessel. The temperature of liquor inside the vessel is therefore more consistent, still further improving the efficiency of conversion of starch to fermentable sugars in the system.

Some aspects include a second vessel, wherein the second vessel is a vessel as described above.

In some aspects, the first and second vessels are used to perform the same process from among a mashing process, a sparging process, a boiling process or a fermentation process. This allows the system to increase the volume of output from a given system without reducing efficiency.

In some aspects, the first and second vessels are used to perform the same process from among a mashing process, a sparging process, a boiling process and a fermentation process. The system can therefore have a first vessel at one stage of the brewing process and the vessel at a second stage of the brewing process. For example, the first vessel may be involved in a sparging process while the second vessel is involved in a cleaning process. The system can therefore perform multiple processes required for processes associated with a liquid solution of fermentable sugars simultaneously, and reduce or eliminate the down-time of the system.

Various embodiments and aspects of the present invention are described without limitation below, with reference to the accompanying figures.

### BRIEF DESCRIPTION OF THE DRAWINGS

FIG. 1 depicts a vessel;

FIG. 2A depicts a closed hydraulic system with a primary flow path;

FIG. 2B depicts a closed hydraulic system with a secondary flow path;

FIG. 2C depicts an alternative closed hydraulic system with a primary flow path;

FIG. 3A depicts a vessel;

FIG. 3B depicts an alternative vessel with an outlet;

FIG. 3C depicts another alternative of a vessel with an outlet;

FIG. 4 depicts a method of mashing grist;

FIG. 5A depicts a section view through line A-A shown in FIG. 1 of a vessel with a single pair of first and second tubes;

FIG. 5B depicts a section view of an alternative vessel with two or more first tubes inside a second tube;

FIG. 5C depicts a section view of another alternative vessel with two or more pairs of first and second tubes.

### DETAILED DESCRIPTION OF A PREFERRED EMBODIMENT

The present invention relates to a vessel for use in processes associated with a liquid solution of fermentable sugars, particularly in alcoholic liquid production, a method of using the vessel and a closed hydraulic system including the vessel. Preferably, the liquid solution of fermentable sugars is a grain based, such as from barley, corn, wheat, rice, rye, or oats; whether malted or unmalted. Alternatively, the present invention may relate to a vessel for hot liquor enzymatic cleaning, a method of using the vessel for hot enzymatic cleaning and an associated closed hydraulic system. A vessel comprises at least two tubes, including a first tube inside a second tube, with the tubes being inside a container. It is preferred that the first tube and the second tube are parallel. One or more side walls of the first tube are holed, and at least one side wall of the second tube is holed. The holes in the sidewalls of the first and second tubes allow fluid to pass through the sidewalls, but prevent the passage of grist. Grist can be held in a volume between the first tube and the second tube, and liquid can pass from inside the first tube, through that volume and over the grist, and into a volume between the second tube and the container. The side walls of the container can direct fluid to an outlet of the vessel.

As used herein, processes associated with a liquid solution of fermentable sugars include the processes relating to production of alcoholic liquids (for example, a mashing process, a sparging process, a lautering process, a fermenting process etc.), processes relating to production of syrups (including equivalent processes to the processes relating to production of alcoholic liquids) and production of extracts (including equivalent processes to the processes relating to production of alcoholic liquids), such as malt extracts.

The description below focusses on examples in which the processes associated with a liquid solution of fermentable sugars are processes relating to production of alcoholic liquids. It would be apparent to a person skilled in the art that equivalent processes also apply to processes relating to production of syrups and production of extracts.

In an alternative embodiment, the present invention relates to a vessel for use in enzymatic cleaning, a method of using the vessel and a closed hydraulic system including the vessel. The invention may also be used for similar processes wherein any material to be cleaned may be held in a volume between the first tube and the second tube, and enzymatic cleaning liquid can pass from inside the first tube, through that volume and over the material, and into a volume between the second tube and the container. A vessel comprises at least two tubes, including a first tube inside a second tube, with the tubes being inside a container. It is preferred that the first tube and the second tube are parallel. The one or more side walls of the first tube are holed, and at least one side walls of the second tube are holed.

The description below focusses on examples in which the processes associated with a liquid solution of fermentable sugars are processes relating to production of alcoholic liquids. It would be apparent to a person skilled in the art that equivalent processes also apply to processes relating to using an enzymatic liquor to clean a material held in a volume between the first tube and the second tube.

The closed hydraulic system includes a vessel, a liquid tank, a heating device, a pipe system and a pump. The system is a closed hydraulic system capable of continuous circulating flow of liquid through the vessel under hydraulic pressure. This minimises build-up of areas of high viscosity (i.e. there is less chance of areas with higher local sugar content forming), as well as allowing the liquid to be used to agitate the grist. The efficiency of the conversion of starch to fermentable sugars in the system is thereby improved. Additionally, liquid in the closed hydraulic system can be heated by the heat exchanger while it is continuously pumped around the system, and before it is re-introduced into the vessel. The temperature of liquor inside the vessel is therefore more consistent, still further improving the efficiency of conversion of starch to fermentable sugars in the system. The grist is hydraulically agitated (i.e. using consistent liquid flow to agitate the grist) to avoid the need for mechanical stirrers, and the flow direction of the liquid can be reversed to provide additional agitation. The consistent liquid flow can provide variable control over the temperature, pH and pressure difference across the mash in the vessel.

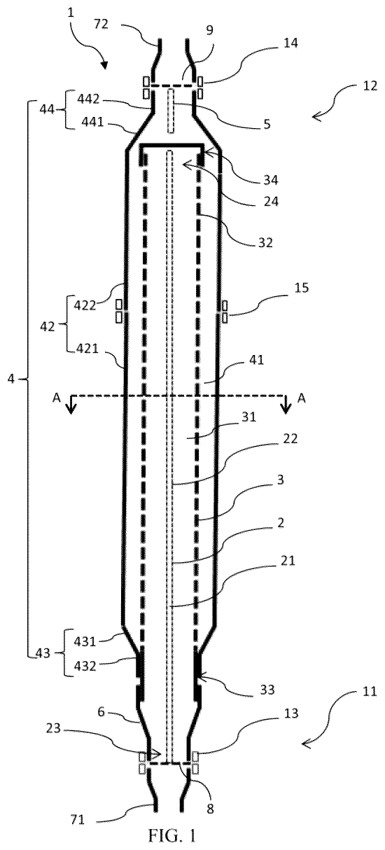

In the arrangement shown in FIG. 1, the vessel 1 for mashing has a first end 11 opposed to a second end 12. A length dimension of the vessel 1 extends between the first end 11 and the second end 12, and a width dimension is perpendicular to the length dimension. In preferred arrangements, the length dimension is greater than the width dimension. Advantageously, having a vessel that is greater in the length dimension than the width dimension may further improve the uniformity of the temperature, pressure and pH of the liquor, and improve the concentration of the liquor by controlling the volume of liquor in the system. In addition, having a vessel 1 that is that is greater in the length dimension than the width dimension allows a reduced footprint for the vessel 1. Furthermore, providing a vessel that is greater in the length dimension than the width dimension, allows for the mash thickness (ratio of liquor to grist) can be controlled by other elements of the closed hydraulic system. Particularly, minimising the distance between the outside wall of at least one second tube 3 and the inside wall of the vessel 1 maximises the control over mash thickness (ratio of liquor to grist) by reducing the minimum amount of liquor required to use the vessel 1 in the below disclosed manner. As such, the amount of liquor in the closed hydraulic system can be chosen and adjusted through other elements of the closed hydraulic system.

The amount of grist, within the second volume 31, through which liquid must pass is limited by the interior of the second tube 3 and the exterior of the first tube 2 in the width dimension. The first tube 2 and the second tube 3 may be arranged such that distance between the exterior of the first tube 2 and the second tube 3 is relatively short. During a mashing process, the liquid therefore travels a relatively short distance through the grist (i.e. the grain bed is relatively thin) compared to conventional flat, mashing tuns. By having a relatively thin grain bed, the pressure differential between the liquid flowing into the vessel 1 and the liquid flowing out of the vessel 1 is reduced. This lowers the chances of areas of high local sugar content forming in the vessel 1, when used in a mashing process, and allows free water in the liquid to reach a higher proportion of the surface of the grain particles in the grist. Enzymatic activity to convert starch to fermentable sugars can therefore occur across a higher proportion of the grist, and the efficiency of the mashing process can be improved. Additionally, the percentage of grist hydraulically agitated (by liquid exiting multiple holes in one or more side walls 21 of the first tube 2) is increased. Advantageously, the vessel 1, and associated closed hydraulic system can achieve a high level of efficiency at a much lower capital cost switching the plant/logistics economics to a point where smaller plants can be used without sacrificing production, which means transport miles and the associated environmental impact can be positively impacted. In some aspects, the vessel 1 is a mashing vessel. In other aspects, the vessel 1 is a mashing and sparging vessel 1. During a sparging process, liquid can reach more of the surface area of the grist (due to the thin grain bed), thereby improving the efficiency of the sparging process and removing the need for a separate lautering tun or mash press.

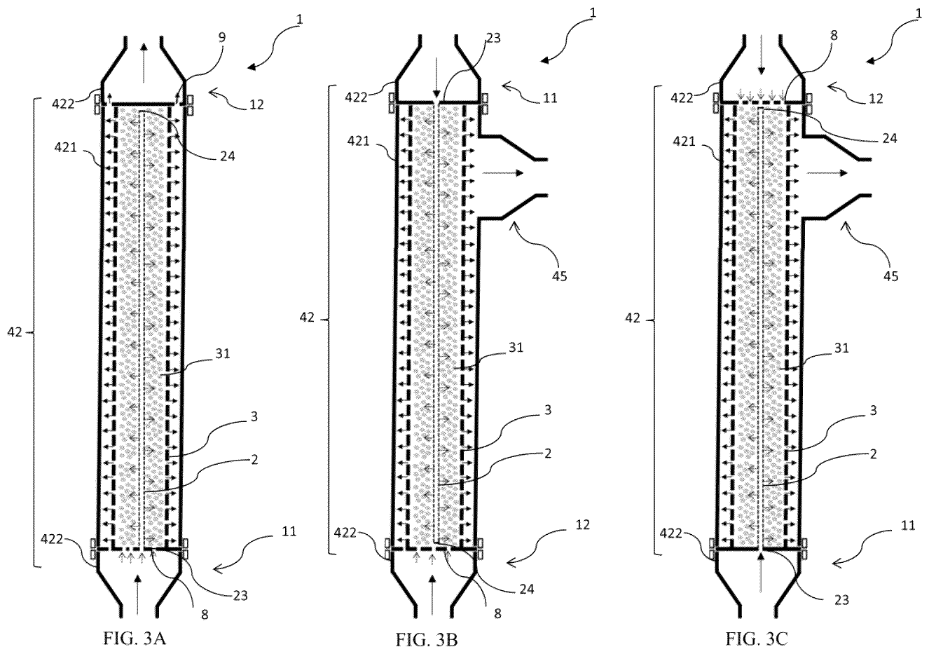

The vessel 1 shown in FIG. 1 comprises a first tube 2, a second tube 3 and a container 4. Preferably, the first tube 2, a second tube 3 and a container 4 are parallel to the axis extending in a length direction between the first end 11 and the second end 12 of the vessel 1. In some arrangements, the first tube 2, a second tube 3 and a container 4 are coaxial to the axis extending in a length direction between the first end 11 and the second end 12 of the vessel 1. In some arrangements, the vessel 1 optionally comprises a third tube 5 located toward an end of the vessel 1 opposed to the first tube 2. A first volume 21 is defined by the interior of the first tube 2, a second volume 31 is defined between the first tube 2 and the second tube 3, and a third volume 41 is defined between the second tube 3 and the container 4. The side wall(s) of the first tube 2 and the second tube 3 are holed to allow the passage of liquid but to prevent the passage of grist. In the arrangement of FIG. 1, the side wall of the container 4 is unholed (i.e. flow of the liquid and grist through the side wall of the container 4 is prevented). In some arrangements, such as is shown in FIG. 3B and FIG. 3C, the side wall of the container 4 is provided with an outlet 45. In such arrangements, the vessel 1 optionally comprises the third tube 5, which is located toward the outlet 45.

In the preferred arrangement, the components of the vessel 1 are made of stainless steel, which will meet standards for production of consumables. At the same time, stainless steel has sufficient tensile strength to withstand the pressure, pH and temperature that arise during a mashing process. Other materials with a suitably high tensile strength can be used, as long as the material meets the pressure requirements, and hygiene requirements (these requirements will change depending on the particular process applied). For example, other possible materials include plastics, iron, copper, aluminium, and brass. As will be understood by a skilled person, different components of the vessel 1 can be made of the different materials.

The first tube 2 shown in FIG. 1 has a uniform cross section in a length direction, the length direction being the longest dimension of the first tube 2 (i.e. the length direction is parallel with a longitudinal axis of the first tube 2, and the longitudinal axis may optionally be the common axis of the second tube 3 and container 4). The first tube 2 extends into the second volume 31 in the length direction, the longitudinal axis extending from the first end 11 of the vessel 1 toward the second end 12 of the vessel 1. The first tube 2 includes one or more side walls 22, having a greatest extent parallel with the longitudinal axis. For example, if the first tube has a circular cross-section, it has one side wall. If it has a square cross section, it will have four side walls.

The second tube 3 shown in FIG. 1 has a uniform cross section in a length direction, the length direction being the longest dimension of the second tube 3 (i.e. the length direction is parallel with a longitudinal axis of the second tube 3, and the longitudinal axis may optionally be the common axis of the first tube 2 and container 4). The second tube 3 extends into the third volume 41 in the length direction, the longitudinal axis extending from the first end 11 of the vessel 1 toward the second end 12 of the vessel 1. The second tube 3 does not touch the container 4 at the second end 12 of the vessel 1.

The second tube 3 includes at least one holed side wall 32, a connected end 33 and a closed end 34. The connected end 33 (also termed an ‘open end’) connects the second tube 3 to the container 4. In some arrangements, the connected end 33 also connects the second tube 3 to the first tube 2.

The closed end 34 (also termed a ‘capped end’) of the second tube 3 is at an end opposite the connected end 33. The closed end 34 is not connected to the container 4. The closed end 34 can be either permanently affixed to the one or more side walls 32 of the second tube 3 or detachably affixed to the one or more side walls 32 of the second tube 3 such that a cap can be detached from the one or more side walls 32. In arrangements where the closed end 34 includes a cap that is detachable from the one or more side walls 32, the cap can be screwed on to the one or more side walls 32 or can be held on by friction. When the cap is held onto the one or more side walls 32 by friction, expansion of the grain bed during operation (e.g. movement of the grist under hydraulic pressure) can lift the cap without detaching the cap from the one or more side walls 32.

The holes in the one or more side walls 32 allow liquid to pass through, thereby directing liquid from the second volume 31 to the third volume 41, but prevent grist from passing through. The holes therefore act as filters to prevent grist from entering the third volume 4 during normal operation of the vessel 1. The size of the holes (e.g. perforations) can vary depending on the crush of the grist intended for the vessel 1. It is preferred that the holes in the side wall 32 of the second tube 3 extend 1 mm or less (and greater than 0 mm) in at least one dimension across a surface of the side wall 32. It is more preferred that the holes are 0.1 mm or greater (i.e. the holes are 0.1 mm to 1 mm) in the at least one dimension. The pattern of the holes on the side wall 32 of the second tube 3 can vary. For example, the pattern can optimise the agitation of the mash as the liquid leaves the second tube 3, or can be simplified for ease of manufacture. The holes may be, for example, perforations or slots, or gaps through a mesh.

The container 4 comprises a tubular section 42, a first end piece 43 and a second end piece 44. In the arrangement shown in FIG. 1, the first end piece 43 tapers inward, toward the longitudinal axis of the container 4 (i.e. the cylindrical axis of the tubular section 42) with increasing distance from the second end piece 44. Also shown in FIG. 1, the second end piece 44 tapers inward, toward the common axis of the co-axial first and second tubes 2, 3 (and optionally container 4) with increasing distance from the first end piece 43. Preferably, the cross section of the tubular section 42 is circular. Optionally, the first end piece 43 includes a drain that, when opened, will allow debris to be removed from the interior of the vessel 1. More optionally, the drain is at the lowest point of the first end piece 43.

The first end piece 43 as shown in FIG. 1 comprises a first frustoconical section 431 portion connected to the tubular section 42 at its largest width, with the smallest width of the first frustoconical portion 431 being sealed around the second tube 3. When the first end piece 43 comprises a frustoconical portion 431, it preferably also includes a first neck portion 432 connected to the frustoconical portion 431 at the smallest width of the frustoconical portion 431. The first neck portion 432 is tubular with a uniform cross section that has the same width as the smallest width of frustoconical portion 431. The tubular section 42 can be sealed to the second tube 3, thereby providing a better seal between the container 4 and the second tube 3 to prevent leakage from the vessel 1.

In use, in the embodiment shown in FIG. 1, the first end 11 of the vessel 1 is located at the bottom of the vessel 1, and the second volume 31 will contain grist. The first tube 2 therefore extends through the grist such that, when liquor passes through the first tube 2 from a pipe 71 coupled to the vessel 1 at the first end 11, it will pass from the first volume 21, through the holes in the first tube 2 and into the second volume 31. In the embodiment shown in FIG. 3B, the first end 11 is located at the top of the vessel 1. In use, in the embodiment shown in FIG. 3B, the first tube 2 therefore extends through the grist such that, when the liquor passes through the first tube 2, it will pass from the first volume 21, through the holes in the first tube 2 and into the second volume 31.

In contrast to a conventional mashing tun, which is large, wide, and operated under ambient pressure, the vessel 1 shown in FIG. 1 is narrow. The distance in a width dimension between the first tube 2 and the inner wall of the second tube 3 is relatively small. As such, there is a maximum distance that the grist can move away from the first tube 2. The size of the second tube 3 and the first tube 2 can therefore be selected to ensure all of the grist is stirred when the liquor is introduced into the vessel 1. The liquor, and temperature and pH associated therewith, is therefore applied to the grist more evenly. This helps ensure that more of the starch in the grist is converted to fermentable sugars, thereby improving the efficiency of the mashing process.

Furthermore, in arrangements according to the present invention, grist is contained in the second volume 31 (i.e. between the first tube 2 and the second tube 3). Liquid passes into the second volume 31 through one or more side walls 22 of the first tube 2, and exits the second volume 31 through one or more side walls 32 of the second tube 3. Liquid can therefore exit the second volume over a greater area than arrangements where liquid that has interacted with grist exits a tube only at an end of the tube (such as in DE202015007481U1 and US2017/0283751A1). Compared to arrangements where the liquid exits a tube only at an end of the tube, arrangements according to the present invention have a lower pressure differential between the first end 11 of the vessel 1 and the second end 12 of the vessel 1 (for example, if the pressure of liquid entering at the first end 11 is 3bar, the pressure of liquid exiting the vessel 1 at the second end may be 2 or 2.5 bar). Particularly, liquid can travel a much shorter distance through the grist (i.e. from the first tube 2 to the second tube 3 rather than some of the liquid having to travel through the entire grain bed from the first end of the vessel 1 to the second end of the vessel 1). The shorter distance of travel through the grist reduces the pressure differential. This, in turn, results in a lower temperature differential across the liquid in the vessel 1. As the temperature of liquid in the vessel 1 is more uniform, it can also be controlled more accurately. Controlling the temperature inside the vessel 1 more accurately means that the interior environment of the vessel 1 can spend more time within desired temperature ranges that correlate to the activation temperature windows of various enzymes in the grist. In arrangements where the first and second tubes 2, 3 are coaxial, liquid will travel a more similar distance through the grist irrespective of the distance from the first end 11 that the liquid enters the second volume 31. Accordingly, the pressure differential and, hence, the temperature differential is further reduced. The width of the tubular section 42 of the container 4 can be selected to minimise the distance between the second tube 3 and the container 4, thereby improving control over the mash thickness (ratio of liquor to grist) by reducing the minimum amount of liquor required to use the vessel 1. Particularly, the mash thickness (ratio of liquor to grist) can be controlled by other elements of the closed hydraulic system. It is preferred that the mash thickness (ratio of liquor to grist) is controlled to be 3 L/kg or greater. It is more preferred that the mash thickness (ratio of liquor to grist) is controlled to be 4 L/kg or less (i.e. that the ratio of liquor to grist is 3 L/kg to 4 L/kg. Selecting a width of the tubular section 42 of the container 4 to minimise the distance between the second tube 3 and the container 4 also increases the uniformity of temperature and pH throughout the vessel 1).

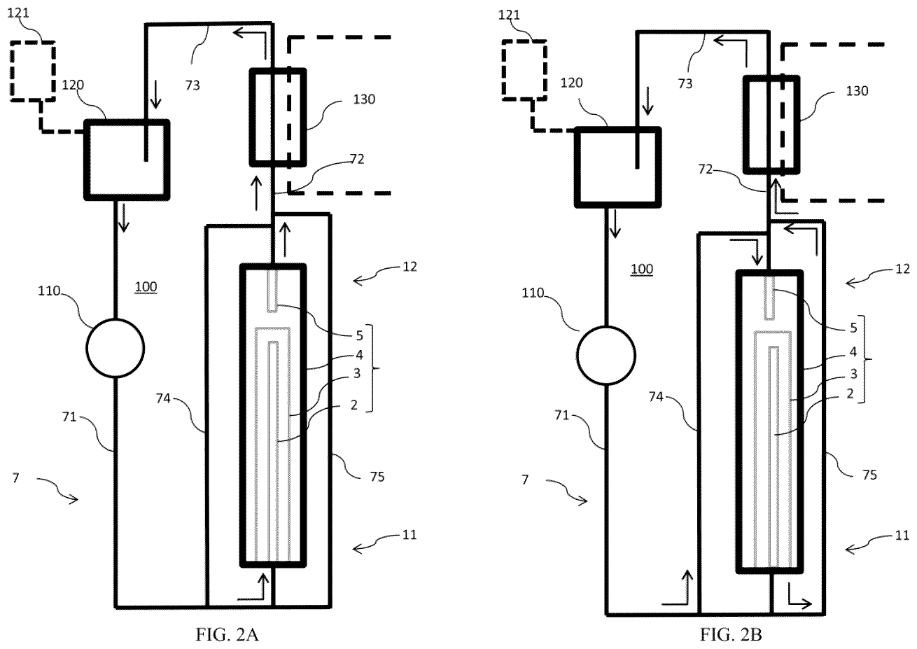

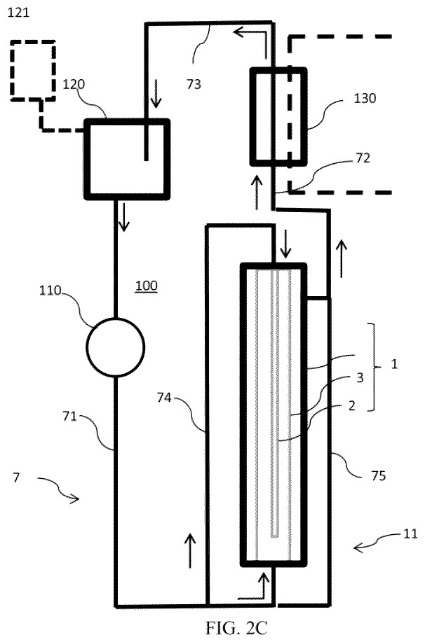

As discussed above, the one or more side walls 22 of the first tube 2 are holed. The one or more side walls 22 are connected to an open end 23 and a closed end 24 of the first tube 2. The closed end 24 is closer to the second end 12 of the vessel 1 than the open end 24. The holes in the one ore more side walls 22 allow the mash to be hydraulically stirred (or otherwise agitated) in the second volume 31, thereby removing the need for mechanical stirrers that are present in conventional mashing tuns. Particularly, when pressurised liquid is fed into an open end 23 of the first tube 2, the holes (e.g. perforations) act as dispersion points. With liquid exiting the first tube 2 via the holes, the liquid acts as a jet, thereby hydraulically agitating the mash. If liquid flows in the opposite direction (i.e. into the first tube 2, rather than out of the first tube 2), the holes act as filters to prevent grist from entering the first tube 2. This may happen when liquid flow through the vessel 1 is reversed (i.e. when liquid enters the vessel 1 through the third tube 3 in the arrangement shown in FIG. 2B). The size of the holes (e.g. perforations) in the one or more side walls of the first tube 2 can vary depending on the crush of the grist intended for the vessel 1. It is preferred that the holes in the one ore more side wall 22 of the first tube 2 extend 1 mm or less (and greater than 0 mm) in at least one dimension across the surface of the first tube 2. It is more preferred that the holes are 0.1 mm or greater (i.e. the holes are 0.1 mm to 1 mm) in the at least one dimension. The pattern of the holes on the one ore more side wall 22 of the first tube 2 can vary. For example, the pattern can optimise the agitation of the mash as the liquid leaves the first tube 2, or can be simplified for case of manufacture. The holes may be, for example, perforations or slots, or gaps through a mesh.

During a mashing process, grist is located in the second volume 31 (inside the second tube, but outside of the first tube). Liquid (i.e. liquor, during a mashing process) constantly flows from the first volume 21 into the third volume 41 via the second volume 31. Particularly, the liquid passes from the first volume 21, through the holes in the one ore more side walls 22 of the first tube 2, and into the second volume 31. The liquid then passes from the second volume 31, through the holes in the side walls of the second tube 3, and into the third volume 41. As the holes in the first tube 2 and the second tube 3 allow the passage of liquid but not the passage of grist, the grist is contained in the second volume 31 as liquid enters the vessel 1 through the first tube 2.

As the liquid exits the first tube 2 under pressure, it causes movement in the grist. In effect, the pressurised liquor entering the vessel 1 through the first tube 2 stirs the grist as the particles of grist closest to the first tube 2 are pushed away, creating a void into which other grist particles can fall and/or float. Introducing the liquid into the vessel 1 under pressure therefore removes the need for mechanical stirrers found in commercial mashing tuns.

In the arrangement shown in FIG. 1, the vessel 1 include an optional reducer 6 that at one end is sized to fit over, and seal to, the second tube 3 and to reduce in width to allow a first connector 13 to connect the reducer 6 to a pipe 71 of a system of piping 7. In other arrangements, the first connector 13 connects the pipe 71 directly to the second tube 3. In still other arrangements, the container 4 extends further from the second end 12 of the vessel 1 than the second tube 3, and the first connector 13 connects the pipe 71 directly to the container 4. In some embodiments, the first connector 13 is a flange arrangement to attach the first pipe 71 to the vessel 1 (e.g. connecting the first pipe 71 to the second tube 3, the container 4 or the reducer 6, depending on the specific arrangement of the vessel 1).

The vessel 1 may include a first holed disc 8 defining a boundary of the second volume 31. The holes in the holed disc 8 allow liquid to enter the second volume 31. The first holed disc 8 disperses the liquid, under hydraulic pressure, into as many parts of grist lowest in the second volume 31 (i.e. the lower grain bed) as possible causing the milled grain particles to float and move. When the first connector 13 is a flanged arrangement, the first holed disc 8 may be located within the flanged arrangement (e.g. sandwiched between two corresponding flanges). In some arrangements, such as is shown in FIG. 1 and FIG. 3A, the first holed disc 8 may define the boundary of the second volume remote from the second end 12 of the vessel 1. In other arrangements, such as is shown in FIG. 3B and FIG. 3C, the first holed disc 8 may define the boundary of the second volume 31 remote from the first end 11 of the vessel 1. In such arrangements, the first holed disc 8 may be connected to the second end piece 44. In arrangements wherein the first holed disc 8 defines the boundary of the second volume 31 remote from the first end 11 of the vessel, liquid may enter the vessel 1 at both the first end 11 of the vessel via the first tube 2 and at the second end 12 of the vessel via the first holed disc 8. In such arrangements, an outlet 45 is provided at a side wall of the container 4 of the vessel 1 for the liquid to exit the third volume 41.

During normal operation, liquid can therefore enter second volume 31 under hydraulic pressure via both the one or more side walls 22 of the first tube 2 and the first holed disc 8. Liquid entering the second volume 31 through holes in the one or more side wall 22 of the first tube 2 will have a have a more horizontal vector than liquid entering the second volume 31 through the holed disc 8. Liquid entering the second volume 31 through the holed disc 8 will have a have a more vertical vector than liquid entering the second volume 31 through the holes in the one or more side walls 22 of the first tube 2. This helps ensure that liquid temperature and pH is consistent within the second volume. At the same time, the holes in the first holed disc 8 assist in agitating mash (for example, continuously floating and moving mash away from the first end 11 of the vessel 1 in a direction perpendicular to the direction of flow of liquid leaving the first tube 2). As the grist in the second volume 31 undergoes more agitation, clumping can be avoided and more surface area of the grist can be exposed to liquid (e.g. liquor), hence increasing the yield of fermentable sugars from the grist in the second volume 31. The size of the holes (e.g. perforations) in the first holes disc 8 can vary depending on the crush of the grist intended for the vessel 1. It is preferred that the holes in the first holed disc 8 preferably extend 1 mm or less (and greater than 0 mm) in at least one dimension across a surface of the disc 8. It is more preferred that the holes are 0.1 mm or greater (i.e. the holes are 0.1 mm to 1 mm) in the at least one dimension. The holes may be, for example, perforations or slots, or gaps through a mesh.

In some arrangements, the first tube 2 can extend through a centre hole of the first holed disc 8, with the holes of first holed disc 8 being at a greater distance in a width dimension from the centre hole of the disc 8 than the one or more side walls 22 of the first tube 2. In such arrangements it is preferred that the first holed disc 8 touches the first tube 2 (e.g. at the one or more side walls 22). In the arrangement shown in FIG. 1, the first holed disc 8 is attached to the end of the reducer 6 remote from the second end of the vessel 1. In such an arrangement, during normal operation, liquid can flow into the second volume 31 through the holes in the first holed disc 8 and through the holes in the first tube 2. In arrangements that do not include a reducer 6, the first holed disc 8 can be attached directly to the second tube 3. In some arrangements, the container 4 extends further from the second end 12 of the vessel 1 than the second tube 3, and the first holed disc 8 can attach directly to the container 4.

In some arrangements the first tube 2 can include a probe that fits inside the first tube 2 from the open end 23 of the first tube 2, and allows measurement of one or more variables of the fluid. It is preferred that the probe is able to measure temperature (i.e. the probe includes a thermocouple or a thermistor). Additionally or alternatively, the probe can measure pH and/or sugar content and/or viscosity. The probe may be a rod. In other arrangements, the probe can be different shapes and sizes. The probe can be either wired or wireless. In some arrangements, the probe is not provided and one or more variables of the fluid can be measured in other ways. For example, thermocouples affixed to the side walls of the vessel 1 or pH and/or sugar content measured from fluid taken from the fluid flow. In some arrangements, the first tube 2, the first holed disc 8 and the probe may be included in a first tube assembly that can be removed from the vessel 1 as a single unit.

FIG. 1 depicts the second end piece 44 that includes a second frustoconical portion 441 connected to the tubular section 42 at its largest width, with the smallest width of the second frustoconical portion 441. When the second end piece 44 comprises a frustoconical portion 441, it preferably also includes a second neck portion 442 connected to the frustoconical portion 441 at the smallest width of the second neck portion 442. The second neck portion 442 is tubular with a uniform cross section that has the same width as the smallest width of frustoconical portion 441. A second connector 14 connects the second end piece 44 to a pipe 72 of the system of piping 7. In some embodiments, the second connector 14 is a flange arrangement to attach the second pipe 71 to the container 4.

In arrangements wherein the first holed disc 8 is disposed at the first end 11, such as is shown in FIG. 1 and FIG. 3A, the vessel 1 may include a second holed disc 9 defining the boundary of the third volume 41 remote from the first end 11 of the vessel 1. The holes in the second holed disc 9 allow liquid to exit the third volume 41 as close to the second end 12 of the vessel 1 as possible. In such arrangements, during normal operation, when liquid enters the vessel 1 at the first end 11, the second holed disc 9 acts as an additional filter in the event that solid matter (e.g. in the event that grist is physically broken into smaller particles during agitation) passes through the holes in the second tube 3 and into the third volume 41. In such arrangements, when the second connector 14 is a flanged arrangement, the second holed disc 9 may be located within the flanged arrangement (e.g. sandwiched between two corresponding flanges). In other arrangements wherein the outlet 45 is provided on the side wall of the container 4, the second holed disc 9 may be provided in the outlet 45 or at the boundary of the outlet 45 and the side wall of the container 4. The size of the holes in the second holed disc 9 can vary depending on the crush of the grist intended for the vessel 1. It is preferred that the holes in the second holed disc 9 are preferably 1 mm or less (and greater than 0 mm) in at least one dimension across a surface of the second holed disc 9. It is more preferred that the holes are 0.1 mm or greater (i.e. the holes are 0.1 mm to 1 mm) in the at least one dimension. The holes may be, for example, perforations or slots, or gaps through a mesh.

Similarly to the first tube 2, the third tube 5 includes one or more side walls, an open end and a closed end. The one or more side walls of the third tube 5 are connected to the open end and the closed end of the third tube 5. The holes in the one or more side walls of the third tube 5 filter liquid exiting the vessel 1 during normal operation. In some arrangements, such as is shown in FIG. 1, the third tube 5 extends further in a length direction than any other direction. The length direction is orientated parallel to a central axis of the first and second tubes 2, 3 and container 4. In such arrangements, the third tube 5 therefore extends into the third volume 41 from the second end 12 of the vessel 1. Having a third tube 5 with one or more holed side walls increases the surface area for filtering liquid as it exits the vessel 1. In turn, this reduces the chances of blockages. The size of the holes (e.g. perforations) in the side walls of the third tube 5 can vary depending on the crush of the grist intended for the vessel 1. It is preferred that the holes in the side wall of the third tube 5 extend 1 mm or less (and greater than 0 mm) in at least one dimension across a surface of the third tube 5. It is more preferred that the holes are 0.1 mm or greater (i.e. the holes are 0.1 mm to 1 mm) in the at least one dimension. The pattern of the holes on the side wall of the third tube 5 can vary. For example, the pattern can optimise the agitation of the mash as the liquid leaves the third tube 5, or can be simplified for ease of manufacture. The holes may be, for example, perforations or slots, or gaps through a mesh. In other arrangements wherein the outlet 45 is provided on a side wall of the container 4, the third tube 5 may extend into the third volume 41 from the outlet 45 in a direction parallel the outlet 45. In such arrangements, the third tube 5 may be connected to the outlet 45 provided at a side wall of the container 4.

In the arrangement shown in FIG. 1, where a second holed disc 9 is present remote from the first end 11, the third tube 5 extends through a centre hole of the second holed disc 9, with the holes of second holed disc 9 being at a greater distance in a width dimension from the centre hole of the second holed disc 9 than the one or more side walls of the third tube 5. It is preferred that the second holed disc 9 touches the third tube 5 (e.g. at the one or more side walls). In arrangements where the closed end 34 of the second tube 3 includes a detachable cap held on to the one or more side walls 32 by friction, the third tube 5 can prevent the cap from detaching from the one or more side walls 32.

Similarly to the first tube 2, the third tube 5 may include a probe that is inside the third tube 5, and allows measurement of one or more variables of the liquid. In some arrangements, the probe is inside the third tube 5 from the second end 12. In other arrangements, wherein the outlet 45 is provided, the probe is inside the third tube 5 parallel to the direction of the outlet 45. It is preferred that the probe is able to measure temperature (i.e. the probe includes a thermocouple or a thermistor). Additionally or alternatively, the probe can measure pH and/or sugar content and/or viscosity. The probe may be a rod. In other arrangements, the probe can be different shapes and sizes. The probe can be either wired or wireless. In some arrangements, the probe is not provided and one or more variables of the fluid can be measured in other ways. For example, thermocouples affixed to the side walls of the vessel 1 or pH and/or sugar content measured from fluid taken from the fluid flow. In some arrangements, the third tube 5, the second holed disc 9 and the probe included with the third tube 5 may be included in a second tube assembly that can be removed from the vessel 1 as a single unit.

Optionally, and as shown in the arrangement of FIG. 1, the tubular section 42 of the container 4 can be separable into a first subsection 421 and a second subsection 422. The first subsection 421 and the second subsection 422 are connected by a subsection connector 15. In some embodiments, the subsection connector 15 includes a flange on the first subsection 421 and a corresponding flange on the second subsection 422. An arrangement in which the container 4 is constructed in subsections allows grist to be more easily introduced and extracted from the second volume 31. For example, the closed end 34 may comprise a cap, which may be detachable from the one or more side walls 32 to allow the second tube 3 to be opened. Once the second subsection 422 has been detached from the first subsection 421 and removed, the cap can then be removed to allow access to the second volume 31. In another example, the second tube 3 can be removable from the vessel 1 to be refilled and replaced. In some such examples, where the first holed disc 8 is directly connected to first end of the second tube 3, the first tube 2, second tube 3 and holed disc 8 may be removed as a single unit. In some arrangements, such as is shown in FIG. 3B and FIG. 3C, the tubular section 42 of the container 4 can be separable into a first subsection 421 and two second subsections 422. Each second subsection 422 may be connected to the first subsection 421 by a subsection connector 15.

As liquid (liquor or wort or sparge water) is fed into the first tube 2, gas (e.g. air) inside the vessel 1 is pushed out of the third tube 5, and bled from the closed hydraulic system 100. The pressure of the liquid entering the vessel 1 through the first tube 2 will dictate the pressure of the gas leaving the vessel 1 through the third tube 5. As such, the vessel 1 may optionally include a bleed valve (not shown) as close to the second end 12 as possible to allow bleeding of gas from the vessel 1. For example, the third tube 5 may include a bleed valve (not shown). In some arrangements, the bleed valve (not shown) is part of an air-bubbler tube, which allows a visual indication when gas no longer passes through the air-bubbler tube. In other optional arrangements, the bleed valve (not shown) is located at the highest point of the closed hydraulic system 100 to avoid air locks in the system 100 and to reduce the risk of hot side aeration taking place. For example, the bleed valve may be included in a pipe of the system of piping 7 either in addition to or instead of the bleed valve of the vessel 1. Such a bleed valve included in a pipe of the system of piping 7 may be part of an air bubbler tube in a similar manner as described above with regard to the bleed valve of the vessel 1.

An example closed hydraulic system 100 using the vessel 1 is shown in FIGS. 2A, 2B and 2C. The system 100 may be a mashing system or may be a mashing and sparging system.

The closed hydraulic system 100 includes a vessel 1 as discussed above, a pump 110, a heating device, and a pipe system 7. The system 100 can additionally include a liquid tank 120. In the preferred embodiment, the heating device is operable to continuously increase the temperature of the liquid up to 80° C. as it is being pumped around the system 100. Advantageously, this eliminates the need for a decoction mashing process and the associated equipment. More preferably, the heating device is operable to continuously increase the temperature of the liquid to 80° C. with varying rates of temperature increase. The heating device shown in FIGS. 2A, 2B and 2C, and discussed herein, is a heat exchanger 130, but other heating devices to heat the liquid circulating around the system 100 can be used. For example, the heating device can be a heating element. In some arrangements, the liquid tank 120 is a heated liquid tank. In some arrangements having a heated liquid tank, the heated liquid tank is the heating device of the closed hydraulic system 100. In other arrangements having a heated liquid tank, the heated liquid tank is in addition to a separate heating device of the system 100. When the vessel 1 is a mashing and sparging vessel, the heating device can be operable to heat liquid (e.g. sparging water) to boiling point, and more preferably to within a temperature range of 70° C. to 80° C. In some arrangements, optional feed tank 121 includes a heating element capable of heating liquid up to 70° C. to 80° C. Advantageously the feed tank 121 can therefore heat liquid to a temperature range of 25° C. to 50° C. prior to the mashing process to assist mixing the liquor blend. Additionally, the temperature range of 70° C. to 80° C. assists washing (rinsing) the fermentable sugars from the grist in the sparging process.

In arrangements such as those shown in FIG. 2A, 2B and 2C, the pipe system 7 includes a first pipe 71 between the liquid tank 120 and the vessel 1. The first pipe 71 is connected to the liquid tank 120 and the vessel 1 and creates a liquid path therebetween. The pipe system 7 further includes a second pipe 72 between the vessel 1 and the heat exchanger 130 (or other heating device). The second pipe 72 is connected to the vessel 1 and the heat exchanger 130 and creates a liquid path therebetween. In arrangements that do not include a liquid tank 120, the first pipe 71 can be connected to the first end 11 of the vessel 1 and the heat exchanger 130.

The pipe system 7 shown in FIGS. 2A, 2B and 2C further comprises a third pipe 73 between the heat exchanger 130 and the liquid tank 120 (when present), a fourth pipe 74 between the first pipe 71 and the second pipe 72, and a fifth pipe 75 between the first pipe 71 and the second pipe 72. Valves (not shown) are provided between fourth pipe 74 and the first pipe 71, between the fourth pipe 74 and the second pipe 72, between fifth pipe 75 and the first pipe 71 and between the fifth pipe 75 and the second pipe 72 to control the flow path (flow direction through the vessel 1). When liquid travels in a primary route around the closed hydraulic system 100, as shown by the arrows in FIG. 2A and 2C, the valves attached to the fourth pipe 74 and fifth pipe 75 are closed. Liquid (liquor or wort or sparge water) therefore travels through the vessel 1 in a first direction. When liquid travels in a secondary route around the system 100, as shown by the arrows in FIG. 2B, the valves attached to the fourth pipe 74 and fifth pipe 75 are open. Liquid therefore travels through the vessel 1 in a second (reverse) direction. In some arrangements, the fifth pipe 75 can connect to the heat exchanger 130 instead of connecting to the second pipe 72. In some arrangements, the fifth pipe 75 can connect to the vessel 1 instead of connecting to the first pipe 71.

In the preferred arrangements, the liquid tank 120 is higher than the top of the vessel 1. This allows liquid from the liquid tank 120 to be fed into the vessel 1 under gravity to gently remove gas from the vessel 1. In some arrangements, this can be pump assisted.

Preferably, and as in the arrangement shown in FIGS. 2A, 2B and 2C, the pump 110 is connected in the first pipe 71.

The heat exchanger 130 can raise the temperature of the liquid travelling around the primary or secondary liquid paths in a continuous manner. Minerals or salts can be added into the liquid in the liquid tank 120 in order to adjust the pH of the liquid.

FIGS. 2A, 2B and 2C also show an optional liquor feed tank 121. The liquor feed tank 121 can be used to introduce liquor into the liquid tank 120 to maintain a target liquor-grist ratio. During a mashing process, feed 121 can be isolated from the liquid tank 120. Sparge water to be used during a sparging process, to rinse the fermentable sugars from the grist and increase the proportion of fermentable sugars collected from the grist, can then be introduced into the feed tank 121. Once the mashing process is complete, the sparge water can be introduced from the feed tank 121 into the liquid tank 120.

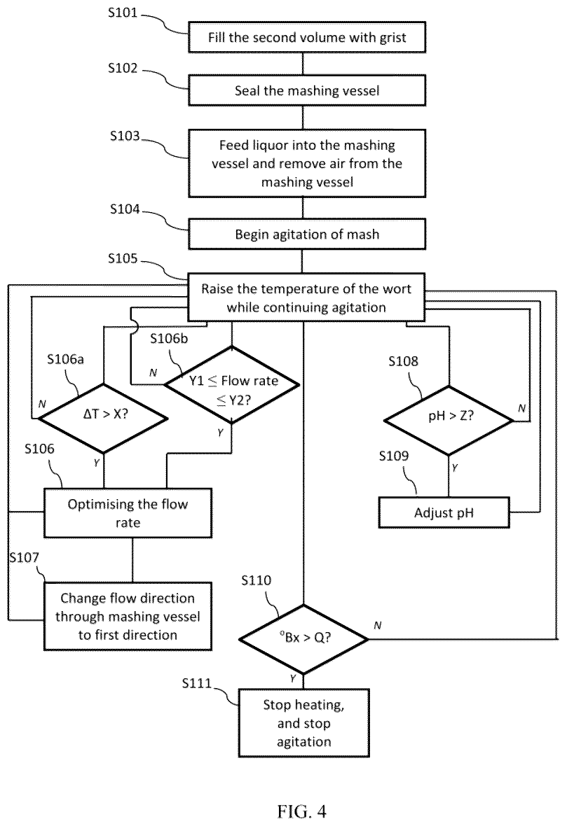

A mashing process will now be described with reference to FIG. 4. The method is discussed using the system 100, but can apply to other systems. At step S101, the vessel 1, is filled with grist. More particularly, the second volume 31 is filled with grist. The second volume 31 may be filled while the second tube is located within the container 4 through an opening in the container 4 (such as may result from detaching the second end 44 if the second end 44 is detachable, or by detaching the first subsection 421 from the second subsection 422 if they are present in the arrangement, or by disconnecting the pipe 72 from the second end 44 and removing the third tube 5 and second holed disc 9 if present). Alternatively, filling the second volume 31 with grist can be done while the first tube 2 and second tube 3 are separated from the container 4 as a single removable unit (for example, the first tube 2 and second tube 3 may be a cartridge removable from the container 4). If the vessel 1 includes a first holed disc 8, and the first holed disc 8 is attached to the second tube 3, filling the second volume 31 with grist may occur when the first tube 2, second tube 3 and first holed disc 8 are separated from the container 4 as a single removable unit (in the above mentioned example, the cartridge may contain the first tube 2, second tube 3 and the first holed disc 8). In arrangements where the first tube 2 and second tube 3 are removed from the container 4 to fill the second volume 31, the single removable unit (optionally also containing the first holed disc 8) can be a disposable unit or may be a re-fillable unit (either on-site or off-site).

Separating the removable unit from the container 4 may include detaching the first subsection 421 of the container 4 from the second subsection 422 of the container 4. In other arrangements, the second end piece 44 can be detached from the tubular section 42 to allow the removable unit to be removed from the container 4 and filled.

In arrangements such as that shown in FIG. 1, the closed end 34 of the second tube 3 includes a cap which may be detachable to allow grist to be placed within the second volume 31. Alternatively, the second tube 3 can be inverted to allow grist to be placed within the second volume from the open end 33 of the second tube 3.

At step S102, the second volume 31 and the vessel 1 are sealed. Sealing the second volume 31 will depend on how the grist is placed within the second volume 2. For example, if the closed end 34 includes a removable cap, such that grist can enter the second volume 31 through the closed end 34 of the second tube 3, sealing the second volume 31 includes reattaching the cap.

Sealing the vessel 1 includes re-attaching the first subsection 421 to the second subsection 422, if the vessel 1 includes the first subsection 421 to the second subsection 422 and they were detached to fill the second volume 31 with grist. Sealing the vessel 1 may include re-attaching the first subsection 421 to each second subsection 422, if the vessel 1 includes the first section 421 and two second subsections 422 and they were detached to fill the second volume 31 with grist. Sealing the vessel 1 may also include re-attaching the second end piece 44 to the tubular section 42, if they were detached to fill the second volume 31 with grist. Sealing the vessel 1 may also include re-attaching the second pipe 72 to the second end piece 44, if the second pipe 72 is detached from the vessel 1 to fill the second volume 31 with grist.

At step S103, liquor is fed into the vessel 1 through the first tube 2 to form the mash. The liquor can enter the vessel 1 under gravity and without being heated or cooled—i.e. at ambient temperature (typically 18° C. to 22° C. in a temperate climate). For example, a liquid tank 120 can be held at a height greater than the highest point of the vessel 1, and a valve between an exit aperture of the liquid tank 120 and the first end of the vessel 1 can be opened. In some arrangements, a pump 110 can be used to adjust the pressure of liquor entering the vessel 1. The pump 110 can be used to assist a gravity-fed arrangement, or can be used on its own in arrangements that aren't gravity-fed.

Advantageously, feeding the liquor into the vessel 1 at ambient temperature and under gravity gently introduces liquid into the vessel 1 while removing the air without warming the liquid. The temperature of the liquor at this point is below the Hot-Side Aeration (HSA) minimum of 26 degrees Celsius. As liquor is fed into the vessel 1 at a temperature below 26 degrees Celsius, introduction of inert gases to avoid HSA is not necessary, and the efficiency of the mashing process can be improved.

As the liquor enters the vessel 1 (e.g. through the first pipe 2), air inside the vessel 1 is forced toward the top of the vessel 1 (i.e. directly at the top or near the top of the vessel 1). Step S103 can also therefore include releasing gas (e.g. air) from the vessel 1 through a bleed valve. Once all the gas has left the vessel 1, the bleed valve is closed. Optionally, the bleed valve is part of an air-bubbler tube (not shown). Optionally, the bleed valve is at the second end 12 of the vessel 1. Alternatively, the bleed valve is part of the system 100 external to the vessel 1 and preferably at the highest point of the system 100. When an air bubbler tube is used, gas leaving the vessel 1 is directed through a liquid thereby allowing a user to see bubbles while gas leaves the vessel 1. Once all the gas has left the vessel 1, the bubbles will no longer be visible and the bleed valve is closed. If gas is released through the bleed valve during step S104, liquor can be added to keep the amount of liquid constant. For example, a feed tank 121 can be included to introduce liquor into the system 100. In some arrangements, the feed tank 121 is a liquor holding tank to top-up the liquid level in the liquid tank 120.

When a pump 110 is used to assist a gravity-fed arrangement, it can be used to complete oxygen removal from the vessel 1. For example, liquor can be transferred from the liquid tank 120 into the vessel 1 under gravity before the pump 110 is activated. Activation of the pump 110 can be in response to one or more factors including: a determination that a preset time period has elapsed, a determination that a preset amount of liquor has been fed into the vessel 1, a determination that the amount of liquor in the liquid tank is less than a predetermined level, a determination that the mash in the vessel 1 is above a predetermined level (i.e. the highest point of the mash is above the predetermined level), and a determination that the flow-rate of gas through the bleed valve is less than a predetermined flow rate.

As some of the enzymes in the grist have an activation range starting at 20° C. (e.g. Beta-glucanase, Proteases, Peptidases), feeding liquor into the vessel 1 may start the mashing process. However, in order to improve the yield, a hydraulic mash agitation process begins at step S104.

In the preferred embodiment, hydraulically agitating the mash includes using a pump 110 to force liquor to follow a primary path around the system 100 (the arrows in FIG. 2A and FIG. 2C indicate a primary path according to a preferred embodiment). This causes the liquid to flow through the vessel 1 in a first direction; liquid enters the first volume 21 in the first tube 2, before passing through the holes in the first tube 2 and into the second volume 31. When the vessel includes a first holed disc 8, liquid can also enter the second volume 31 through holes in the first holed disc 8.

Liquid in the second volume 31 then passes through the holes in the second tube 3, and into the third volume 41. In arrangements wherein the first holed disc 8 is provided at the first end 11, once in the third volume 41, the liquid will be directed toward the second end 12 of the vessel 1, as the side walls of the container 4 are impervious to liquid. At the second end 12 of the vessel 1, the liquid exits the vessel 1 through the second end piece 44. In other arrangements, wherein the outlet 45 is provided at a side wall of the container 4, the liquid exits the vessel 1 through the outlet 45. In arrangements that use a pump to feed liquor into the vessel 1 to create mash and/or remove air, the same pump 110 can be used to cause the liquor to flow to thereby agitate the mash.

The initial agitation ensures that any remaining oxygen or air in the vessel 1 (for example, mixed with the grist) in the closed hydraulic system 100 is removed. While the action of the pump, and motion of the liquor, will cause the temperature to rise slightly, it will still be below 26 degrees Celsius at this point. The initial agitation of the mash is therefore carried out below the HSA minimum temperature.

As the mashing process has now begun, the liquid that exits the vessel 1 will continue along the first path to eventually re-enter the vessel 1. Therefore, the liquid can be continuously moving, circulating through the vessel. As such, it is less likely that areas of high viscosity (i.e. areas of high local sugar content) are formed. Therefore free water is more evenly distributed throughout the grain bed, thereby improving control of the conditions in the vessel and allowing enzymatic conversion to occur across the grain bed in a more uniform manner to improve efficiency of conversion of starch into fermentable sugars. In addition, being able to continuously circulate liquid through the vessel allows the liquid to reach as much of the surface area of the grist as possible without the need for mechanical stirrers.

When hydraulically agitating the mash, the pump 110 feeds liquid (liquor or wort) through the first tube 2 at a higher pressure than during creation of the mash and/or removal of air in step S103. The higher pressure is dependent on a number of factors, including the size of the vessel 1, the grist being used, the liquid being used and the temperature of the liquid. For example, the higher pressure can be at least 1bar. In some examples, the higher pressure is from 1bar up to 25bar. The higher pressure liquid exiting the first tube 2 causes the mash to move within the vessel 1. Hydraulically agitating the mash in this manner removes the need for mechanical stirrers and ensures even temperature distribution throughout the grist.

While production of wort has begun, different enzymes in the grist activate in different temperature ranges. Accordingly, in order to improve the yield of sugar released in the mashing process, the temperature of the liquor is continuously increased while continuing to pump the liquid around the closed hydraulic system 100 at step S105. The continuous increase in temperature can be at varying rates, such that temperature increases at a different rate at different times within the mashing process. For example, the rate of temperature increase may be reduced in temperature ranges associated with the highest activation period of certain enzymes (i.e. the period associated with the highest rate of conversion of starches to fermentable sugars for the certain enzymes). Similarly, the flow rate of liquid through the vessel 1 can be increased when the temperature of the liquid is in a range associated with the lowest activation period of certain enzymes.

In a preferred arrangement, a heat exchanger 130 is used to raise the temperature of the wort in the liquid circuit. The heat exchanger 130 is preferably part of a heat exchange control system that includes a first series of pipes, through which liquid from the vessel 1 can flow, and a second series of pipes, through which a temperature control liquid can flow. The first series of pipes are adjacent to the second series of pipes, such heat can be exchanged between the liquids in the first and second series of pipes. Accordingly, adjusting the temperature of the temperature control liquid will adjust the temperature of the liquid from the vessel 1.

The temperature is raised in a continuous manner as the wort is pumped around the closed hydraulic system 100. In contrast to the discrete, step-wise temperature increase of conventional arrangements, the continuous increase in temperature improves the chances of activating each enzyme group optimally, as the temperature level thereby increases the amount of fermentable sugar released by the grist.

During the mashing process, the viscosity of the mash can change (due to, e.g. chemical processes or simply from the amount of sugar). This can cause portions of the mash to be stirred more than others, for example when grist particles stick together. This can lead to some sections of the mash being heated more than others and can reduce the surface area of grist that can be penetrated by liquid. To separate any clumps of grist, and to reduce the temperature variation through the mash in the vessel 1, the flow of the wort into the vessel 1 can be reversed (step S106). Particularly, in step S104, the pump 110 initially causes liquid to flow through the vessel 1 containing grist in a first direction. In arrangements such as shown in FIG. 2A, the liquid is pumped into the vessel 1 through the first tube 2. In arrangements such as shown in FIG. 2C, the liquid is pumped into the vessel 1 through the first tube 2 and through the first holed disc 8 provided at the second end 12. The liquid will then exit the vessel 1 through the third tube 5. The liquid therefore initially travels through the vessel 1 in a first direction.

Once the agitation of the mash has begun, the flow rate can be optimised (S106). Particularly, action may be taken to control the flow rate within a target range (S106b). Optimising the rate of flow can be in response to a number of factors, and sensors in the closed hydraulic system 100 can provide various indications that the flow rate of liquid through the vessel 1 has reduced (for example, a higher differential pressure between the first pipe 71 and the second pipe 72) or increased. Reduction in that flow rate can be an indication that clumps of grist have formed in the vessel 1, such that liquid flow is hindered. For example, the flow rate of wort leaving the vessel 1 can be directly measured (e.g. optional step 106b).

For example, if the flow rate of wort leaving the vessel 1 is determined to be within the target range, Y1≤Flow rate≤Y2, hydraulic agitation of the mash and increase in temperature S105 continues.

If the flow rate of wort leaving the vessel 1 is determined to be less than the lower value of the target range, Y1, action can be taken to optimise the flow rate. For example, the flow direction can be reversed temporarily (i.e. the flow direction is changed to the second direction, before reverting to flow through the vessel 1 in the first direction). The predetermined range is dependent on the specific geometry of the component of the system, and is selected to show a statistically significant drop off or increase in the flow rate.

When the flow is reversed, the pump 110 causes the liquid through the vessel 1 in a second direction, which is opposite to the first direction. In arrangements such as that shown in FIG. 1, the liquid is therefore pumped into the vessel 1 through the third tube 5 (and second holed disc 9 when present in the vessel 1). The liquid exits the vessel 1 through the first tube 2. The liquid therefore travels through the vessel 1 in a second direction as shown in FIG. 2B.

Reversing the flow direction will change the direction of the force applied on the mash by the liquid being introduced into the vessel 1. Accordingly, clumps of grist or other portions of mash that are not being agitated as expected (e.g. if a vortex forms) when the flow is in the first direction will be moved and agitated when the flow is in the second direction.

In another example, a higher temperature differential between wort at the point of entry to the vessel 1 (e.g. at a temperature sensor inside the first tube 2, such as a temperature sensor on the interior of a side wall of the first tube 2) and wort at the point of exit from the vessel 1 (e.g. a temperature sensor inside the third tube 5, for example a temperature sensor on the inner wall of the third tube 5) can indicate that grist has started to clump. Particularly, when grist clumps, liquid flow through the vessel 1 is hindered. If the heated liquid enters the vessel 1 at the first end 11 (i.e. via the first tube 2 and any associated holed disc 8), even distribution of the heat is promoted by the movement of liquid through the vessel 1. If the liquid flow is hindered (e.g. by clumps of grist), there will be reduced heat transfer to the second end 12 of the vessel 1, and the temperature differential will increase. As such, in optional step S106a, the temperature of wort at the point of entry to the vessel 1 and the temperature of wort at the point of exit from the vessel 1 are measured, the difference, ΔT, between those temperatures can be monitored. If it is determined that the difference, ΔT, between those temperatures is less than predetermined range, X (e.g. 2° C.), hydraulic agitation of the mash and increase in temperature S105 continues. If it is determined that the difference, ΔT, between those temperatures is greater than predetermined range, X (e.g. 2° C.), the flow direction can be reversed temporarily (i.e. the flow direction is changed to the second direction, before reverting to flow through the vessel 1 in the first direction). In some embodiments, both the temperature difference (between wort entering the vessel 1 and wort exiting the vessel 1) and the flow rate (of wort exiting the vessel 1) are monitored. In such embodiments, that include steps 106a and 106b, the change in direction can be in response to either the temperature difference exceeding a predetermined range or the flow rate being below a predetermined lower value, or a combination of both. Examples above indicate that the predetermined range, X, can be 2° C. In other examples, the predetermined range, X, can be between 2° C. and 5° C., for example, 3° C. or 5° C.

For another example, if the flow rate of wort leaving the vessel 1 is determined to exceed the upper value of the target range, Y2, optimising the flow rate (S106) may include throttling the flow rate into or out of the vessel 1 via the first tube 2 and/or the first holed disc 8.

Optimising the flow rate (S106) may include balancing the rate of flow of liquor being fed into the vessel 1 via the first tube 2 with the rate of flow of liquor being fed into the vessel 1 via the first holed disc 8. In another example, optimising the flow rate (S106) may include balancing the rate of flow of liquor being fed into the vessel 1 with the rate of flow of liquor exiting the vessel 1. In those examples, balancing the inflow and outflow of liquor may promote an even flow across the grain bed.

In examples where S106 has included changing the flow direction through the vessel 1, (e.g. from the first direction to the second direction), it can revert back to the original flow direction (e.g. change back to the first direction) in step S107. This can be after a pre-determined time, or when it is detected that the cause for switching directions at step S106 has been removed. The pre-determined time is preferably less than a minute (i.e. greater than 0 seconds but less than or equal to 60 seconds). For example, if the flow direction was changed due to ΔT being greater than a predetermined temperature, X, step S107 may occur when ΔT is no longer greater than X. Similarly, if the flow direction was changed due to the flow rate being less than a predetermined flow rate, Y, step S107 may occur when the flow rate is no longer less than Y. Raising the temperature of the wort (step S105) can occur concurrently with any or all of steps S106, S106a S106b and S107. In other embodiments, the temperature rise can stop while the flow direction is reversed (step S106) and then reverted (step S107). It will be apparent that changing the flow direction and then reverting the flow direction in steps S106 and S107 can be pulsed if, once the flow has reverted, it is determined that further agitation is required. In examples where S106 has not included changing the flow direction through the vessel 1, step S107 is omitted.

In optional step S108, it is determined if the pH of the wort is outside of a target range (also termed a predetermined range), Z. The target range will depend on the desired qualities of the final product and the current temperature in the mashing process. In the preferred embodiment, the pH of the wort is measured at the point of exit from the vessel 1. In other embodiments, the pH can be measured in other locations within the system 100. When it is determined that the pH of the wort is within a target range, Z, hydraulic agitation of the mash and increase in wort temperature S105 continues without adjusting the pH. When it is determined that the pH of the wort is outside of the target range, Z, the pH can be adjusted in addition to continuing agitation and heating of the wort (step S109).

Adjusting the pH can be done in any conventional manner, including adding minerals and salts to the wort. In some embodiments, minerals and salts are added in the liquid tank 120, and will then be carried into the vessel 1 as the wort is pumped around the closed hydraulic system 100. Adjusting the pH by adding minerals or salts to the wort that is used to agitate the mash while the mash is being agitated allows small adjustments to the pH to be achieved very quickly throughout the mash. Accordingly, the pH of the wort can more easily be adjusted to match the activation window for each enzyme as needed, and the yield of fermentable sugar is increased.

In step S110, it is determined if the sugar content (degrees Brix, °Bx) of the wort is above a predetermined level, Q. That predetermined level will depend on the specific grain used to make the grist. To get the maximum yield of fermentable sugars from the grist, the sugar content will exceed the predetermined level when the temperature of the wort is slightly above the denaturing temperature of the enzymes with the highest upper-limit to their activation range (i.e. when enzymes that require the most temperature to release sugars begin to denature). When it is determined that the sugar content of the wort is less than the predetermined level, hydraulic agitation of the mash and increase in the temperature of the wort continues (S105). When it is determined that the sugar content of the wort exceeds the predetermined level, heating the liquid is stopped (step S111). At this point, the mashing process is complete. Agitation of the liquid can continue. This may be desirable if the end of the mashing process immediately precedes the start of a sparging process. Alternatively, agitation of the mash can also be stopped. This will involve stopping the pump from pumping liquid around the closed hydraulic system.

The method shown in FIG. 3 allows the mash to be hydraulically agitated using liquid in the closed hydraulic system 100, thereby removing the need for mechanical stirrers. Further, hydraulically agitating the mash by forcing wort through the mash with a continuous control of temperature as described in the method of FIG. 3 ensures that the temperature of the wort increases through the activation range of each enzyme in the grist.

Preferably, the closed hydraulic system 100 is a mashing and sparging system in which a sparging process begins after the mashing process is complete. In such arrangements, the vessel 1 is a mashing and sparging vessel 1. In other arrangements, the grist and wort from the system 100 can be transferred to a separate sparging tun.

The sparging process begins after completion of the mashing process. The sparging process includes passing the wort around the system 100 and into the vessel 1 to rinse the grist. The wort is then removed from the vessel 1, for example at the second end 12. In preferred arrangements, sparge water is pumped through the system 100 while continuously topping up the liquid tank 120. It is most preferred that the sparge water is pumped in the first direction (i.e. the direction shown in FIG. 2A), although in other embodiments the sparge water can be pumped in the second direction (i.e. as shown in FIG. 2B). Alternatively, the point in the liquid tank 120 through which liquid exits the tank 120 under gravity is higher than a wort drain, through which wort is to exit the vessel 1. This allows the sparging process to occur under gravity feed of sparge water.

In other arrangements, the wort drain is a wort release tap (not shown) at the second end 12 of the vessel 1. In still other arrangements, the wort drain may be a wort release tap (not shown) on the second pipe 72.

In some arrangements, wort can initially be removed from a drain at the second end 12 of the vessel 1. Once the sugar content of the wort removed at the second end 12 of the vessel 1 has dropped below a predetermined level, additional wort can be drained from the vessel 1 at the first end 11 under gravity. For example through a separate drain (not shown).

As wort is collected from the vessel 1, the amount of liquid in the system 100 will be reduced. Sparge water can be added to the system 100, e.g. to tank 120. The sparge water is preferably between 70° C. and 80° C. The sparge water is then introduced into the vessel 1. As with a conventional sparging process, the sparge water rinses the grist of fermentable sugars, thereby increasing the yield of fermentable sugars. However, with the closed hydraulic system 100, the sparge water is passed through the vessel 1 with hydraulic pressure. Accordingly, the fermentable sugars are quickly and efficiently rinsed from the grist.

As sparge water is introduced into the vessel 1, the percentage of fermentable sugar in the liquid exiting the vessel 1 will reduce. The sugar content (Brix Content) of the wort exiting the vessel 1 can be monitored during the sparging process and, when that sugar content falls below a predetermined level, the sparging process stops.

The vessel 1 (whether used for just mashing or for mashing and sparging) shown in FIG. 1 can be cleaned by opening the first end 11 of the vessel 1 (e.g. by removing the first pipe 71 and, when present, the first holed disc 8. Optionally, the first tube 2 may also be removed from the vessel 1 during a cleaning process). The spent grain (e.g. remaining grist and liquid) inside the vessel 1 can then fall into the collection bucket. The remaining liquid from the pipe system 7, the heat exchanger 130 and the liquid tank 120 (if present) can also drain into the collection bucket. At this point, any system filters can be removed and cleaned. Cleaning liquid may be continually pumped through the system 100 in the second (reverse) direction during the cleaning process.

The system 100 is then reconnected, such that liquid can enter the vessel 1 though the first pipe 71 and exit the vessel 1 through the second pipe 72. Any filters that were removed are replaced. Cleaning water is then introduced (e.g. into the liquid tank 120), and pumped around the system 100. In some arrangements, the temperature of the cleaning water is increased in order to improve the cleaning process. The heat exchanger 130 can be used to increase the temperature of the cleaning water. Sterilisation chemicals can be added to the cleaning water, and the temperature can be adjusted depending on the chemicals used. For example, sterilisation chemicals can be put in the liquid tank 120. If needed, the system 100 can remain in a dormant state (i.e. with the pump 110 and heat exchanger 130 turned off) with the sterilisation chemicals therein until it is required for another mashing process.

After the sterilisation chemicals have been added, they are drained from the first end of the vessel 1. In arrangements such as shown in FIG. 1, this can be achieved by disconnecting the first pipe 71, which can then be reconnected to the vessel 1, and hot water can be pumped around the system 100 to rinse the remaining chemicals from the interior surfaces of the system 100. The first pipe 71 can again be disconnected from the first end 11 of the vessel 1, and the hot water can be drained into a collection bucket. In some aspects, the first pipe 71 is re-attached to the vessel 1, and the interior of the system 1 can be rinsed with hot water again. Rinsing the vessel 1 and removing the water in this manner can be repeated as often as needed. Once the sterilisation chemicals have been removed, another mashing process can begin. As an alternative a valve may be provided to drain the chemicals and hot water from the vessel 1.

### Other Aspects, Embodiments and Modifications

The arrangements above discuss the same pump 110 being used to initially introduce liquor into the vessel 1 (step S103) and to hydraulically agitate the grist (step S104). In other arrangements, one pump can be used to initially introduce the liquor into the vessel 1 and another pump can be used to hydraulically agitate the grist.

In arrangements discussed above, a heat exchanger 130 is used to increase the temperature of liquid in the system 100. In other arrangements, other heating means can be used. For example, an immersion heater can be placed in the liquid tank 120, or a heating coil can be placed around one or more of the pipes.

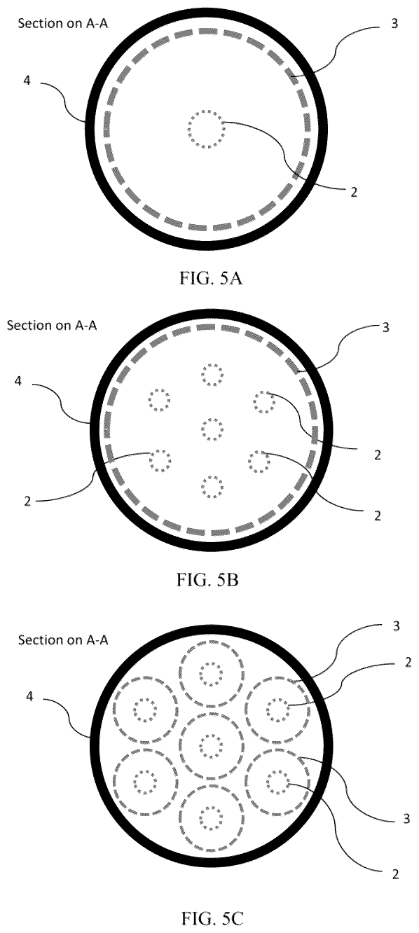

The vessel 1 shown in FIG. 1 and FIG. 5A includes a single pair of tubes (i.e. a first tube 2 and second tube 3) in a container 4. In other arrangements, such as that of FIG. 5B, the vessel 1 can include two or more first tubes 2 inside a single second tube 3 within container 4. In such arrangements, the second tube 3 and the vessel 1 may be differently proportioned, for example with the width dimension being closer in size to the length dimension or with the width dimension exceeding the length dimension, depending on the number of first tubes 2. Such arrangements may allow for greater efficiency for the processes associated with a liquid solution of fermentable sugars while maintaining the advantage of the present invention.

In those arrangements, the two or more first tubes 2 may be arranged such that the distance in a width dimension between the each first tube 2 and the inner wall of the second tube 3 or the outer wall of an adjacent first tube 2 remains relatively small. As such, there is a maximum distance that the grist can move away from each first tube 2. The size of the two or more first tubes 2 and the second tube 3 can therefore be selected to ensure all of the grist is stirred when the liquor is introduced into the third volume 41, such that the liquor, and temperature and pH associated therewith, is applied to the grist more evenly.

Furthermore, this allows those arrangements to maintain a lower pressure differential between the first end 11 of the vessel 1 and the second end 12 of the vessel 1. This, in turn, results in a lower temperature differential across the liquid in the vessel 1. As the temperature of liquid in the vessel 1 is more uniform, it can also be controlled more accurately. Controlling the temperature inside the vessel 1 more accurately means that the interior environment of the vessel 1 can spend more time within desired temperature ranges that correlate to the activation temperature windows of various enzymes in the grist. The width of the vessel 1 can be selected to minimise the distance between the second tube 3 and the container 4, thereby improving control over the mash thickness (ratio of liquor to grist) by reducing the minimum amount of liquor required to use the vessel 1. Particularly, the mash thickness (ratio of liquor to grist) can be controlled by other elements of the closed hydraulic system. It is preferred that the mash thickness (ratio of liquor to grist) is controlled to be 3 L/kg or greater. It is more preferred that the mash thickness (ratio of liquor to grist) is controlled to be 4 L/kg or less (i.e. that the ratio of liquor to grist is 3 L/kg to 4 L/kg. Selecting a width of the vessel 1 to minimise the distance between the second tube 3 and the container 4 also increases the uniformity of temperature and pH throughout the vessel 1.

The vessel 1 shown in FIG. 1 and FIG. 5A includes a single pair of tubes (i.e. a first tube 2 and second tube 3) in a container 4. In other arrangements, such as that of FIG. 5C, the vessel 1 can include two or more pairs of tubes 2, 3 in a single container 4. In such arrangements, the vessel 1 may be differently proportioned, for example with the width dimension being closer in size to the length dimension or with the width dimension exceeding the length dimension depending on the number of pairs of tubes 2, 3. Such arrangements may allow for greater efficiency for the processes associated with a liquid solution of fermentable sugars while maintaining the advantage of the present invention.

In those arrangements, the two or more pairs of tubes 2, 3 may be arranged such that the distance in a width dimension between the each first tube 2 and the inner wall of the each corresponding second tube 3 remains relatively small. As such, there is a maximum distance that the grist can move away from each first tube 2. The size of the two or more pairs of tubes 2, 3 can therefore be selected to ensure all of the grist is stirred when the liquor is introduced into the third volume 41, such that the liquor, and temperature and pH associated therewith, is applied to the grist more evenly.

Furthermore, this allows those arrangements to maintain a lower pressure differential between the first end 11 of the vessel 1 and the second end 12 of the vessel 1. This, in turn, results in a lower temperature differential across the liquid in the vessel 1. As the temperature of liquid in the vessel 1 is more uniform, it can also be controlled more accurately. Controlling the temperature inside the vessel 1 more accurately means that the interior environment of the vessel 1 can spend more time within desired temperature ranges that correlate to the activation temperature windows of various enzymes in the grist. In arrangements where the two or more pairs of first and second tubes 2, 3 are co-axial, liquid will travel a more similar distance through the grist irrespective of the distance from the first end 11 that the liquid enters the second volume 31. Accordingly, the pressure differential and, hence, the temperature differential may remain reduced in arrangements with two or more pairs of tubes 2, 3. The width of the vessel 1 can be selected to minimise the distance between the second tube 3 and the container 4, thereby improving control over the mash thickness (ratio of liquor to grist) by reducing the minimum amount of liquor required to use the vessel 1. Particularly, the mash thickness (ratio of liquor to grist) can be controlled by other elements of the closed hydraulic system. It is preferred that the mash thickness (ratio of liquor to grist) is controlled to be 3 L/kg or greater. It is more preferred that the mash thickness (ratio of liquor to grist) is controlled to be 4 L/kg or less (i.e. that the ratio of liquor to grist is 3 L/kg to 4 L/kg. Selecting a width of the vessel 1 to minimise the distance between the second tube 3 and the container 4 also increases the uniformity of temperature and pH throughout the vessel 1.

In the arrangement shown in FIG. 1, the first end piece 43 includes a frustoconical portion 431 and a first neck portion 432. In other arrangements, the first end piece 43 may include a flat plate with a hole in the middle in place of the frustoconical portion 431. The outer rim of the flat plate connects to the tubular section 44 of the container 4, and the neck portion 432 connects to the flat plate over the hole.

In the arrangement shown in FIG. 1, the second end piece 44 includes a frustoconical portion 441 and a second neck portion 442. In other arrangements, the second end piece 44 may include a flat plate with a hole in the middle in place of the frustoconical portion 441. The outer rim of the flat plate connects to the tubular section 44 of the container 4, and the neck portion 442 connects to the flat plate over the hole.

In the arrangements shown in FIGS. 2A and 2B, a closed hydraulic system 100 includes a single vessel 1. In other embodiments, a closed hydraulic system 100 may include multiple vessels 1. For example, multiple vessels 1 can be connected in parallel. In some examples, some or all of the multiple vessels 1 can be used to perform the same process of an alcohol production method (e.g. the some or all vessels 1 can each be used in a mashing process, in a lautering process, in a sparging process, in a boiling process or in a fermentation process). This allows the closed hydraulic system to increase the amount of output without compromising on efficiency. In other arrangements, some of the multiple vessels 1 can be used to perform different processes of the alcohol production method to provide a continuous output. When multiple vessels 1 are used to perform different processes of the alcohol production method, the closed hydraulic system can perform a mashing process in first vessel 1 while performing a sparging process in a second vessel 1 (and, optionally, a cleaning process in a third vessel 1). Once the mashing process in the first vessel 1 is complete, a sparging process may take place in the first vessel 1; once the sparging process in the second vessel 1 is complete, a cleaning process may occur in the second vessel 1; and once the cleaning process is complete in the third vessel 1, a mashing process may occur in the third vessel. In other examples, the multiple vessels I can be used to perform the same process of a syrup production or extract production. In some embodiments, the system 100 may include three or more vessels 1, with some vessels being connected in parallel and some vessels being connected in series.

In the above description, the vessel 1 is preferably a mashing vessel 1 or a mashing and sparging vessel 1, in which grist (grain) is held in the second volume 31 and a liquid passes through the second volume 31. The vessel 1 may instead be a boiling vessel, in which hops is held in the second volume 31 while wort is pumped around the system 100. The heat exchanger may raise the temperature of the wort to boiling.

In the above description, the vessel 1 is preferably a mashing vessel 1 or a mashing and sparging vessel 1, in which grist (grain) is held in the second volume 31 and a liquid passes through the second volume 31. The vessel 1 may instead be a fermentation vessel 1 in which yeast is held in the second volume 1. When the vessel 1 is a fermentation vessel, wort from a boiling process is cooled before passing through the second volume 31.

In the above description, the vessel 1 is preferably a mashing vessel 1 or a mashing and sparging vessel 1, in which grist (grain) is held in the second volume 31 and a liquid passes through the second volume 31. In some embodiments, the vessel 1 may be suitable for a mashing and sparging, as well as a boiling process and/or a fermentation vessel (i.e. the same vessel 1 may be used for a mashing process and a sparging process, and additionally a boiling process and/or a fermentation process). In such an arrangement, after the sparging process, grist in the second volume 31 will be replaced by hops to be used in a boiling process. After the boiling process, the hops will then be replaced by yeast to be used in a fermentation process.

The description of embodiments, arrangements and aspects has been presented merely for purposes of illustration and description. Suitable modifications and variations to the embodiments and aspects may be performed in light of the above, and different embodiments and aspects may be combined where possible and appropriate, without departing from the scope of protection as determined by the claims.

1. A vessel for use in processes associated with a liquid solution of fermentable sugars, the vessel having a first end and a second end, the second end being opposite the first end, the vessel comprising:
a first tube having an open end, a closed end and one or more holed side walls that extend from the open end to the closed end;
a second tube having at least one holed side wall extending from a connected end of the second tube to a closed end of the second tube, wherein the first tube is located inside the second tube with a volume being defined between the first tube and the second tube; and
a container having a first end piece at the first end of the vessel and a second end piece at the second end of the vessel, wherein the first tube is connected to the container at the open end via the first end piece, and wherein the second tube is located inside and connected to the container at the connected end of the second tube.

2. A vessel according to claim 1, wherein the first tube is parallel with the second tube.

3. A vessel according to claim 1, wherein the second tube is parallel with the container.

4. A vessel according to claim 1, wherein the connected end of the second tube comprises a detachable cap that can be detached from the one or more side wall.

5. A vessel according to claim 4, wherein the cap attaches to the at least one holed side wall of the second tube by friction.

6. A vessel according to claim 1, wherein the first tube is connected to a first pipe connector, the first pipe connector being adapted to connect to a first pipe and to create a liquid path between the first pipe and the first tube.

7. A vessel according to claim 1, wherein holes in the one or more side walls of the first tube are 1 mm or less than 1 mm in at least one dimension across a surface of the side wall.

8. A vessel according to claim 1, wherein the container further comprises a first subsection, at least one second subsection and at least one subsection connector, wherein the first subsection and the at least one second subsection are detachably connected by the subsection connector.

9. A vessel according to claim 1, wherein at least one of the first end piece and the second end piece include a frustoconical portion.

10. A vessel according to claim 1, further comprising a first holed disc connected to the first end piece and the first tube is connected to the first end piece by the first holed disc.

11. A vessel according to claim 10, further comprising a third tube having one or more holed side walls, wherein the third tube is connected to the second end piece and extends toward the first end.

12. A vessel according to claim 11, further comprising a second holed disc connected to the second end piece and the third tube is connected to the second end piece by the second holed disc.

13. A vessel according to claim 1, further comprising a first holed disc connected to the second end piece.

14. A vessel according to claim 1, further comprising a third tube having one or more holed side walls, wherein the third tube is connected to an outlet provided at a side wall of the container.

15. A vessel according to claim 1, wherein the vessel is selected from one or more of the following:
a mashing vessel, operable to hold grain within a volume between the first and second tube; a mashing and sparging vessel, operable to hold grain within a volume between the first and second tube;
a boiling vessel, operable to hold hops within a volume between the first and second tube; or
a fermentation vessel, operable to hold yeast within a volume between the first and second tube.

16. A method of using the vessel according to claim 1, wherein a first volume is defined by an interior of the first tube, the volume defined between the first tube and the second tube is a second volume, and a third volume is defined between the second tube and the container, the method comprising:
feeding liquid into the second volume via the first volume through the one or more holed side walls of the first tube; and
directing liquid from the second volume into the third volume through the at least one holed side wall of the second tube.

17. A method according to claim 16, further comprising determining that the pH of the liquid is outside of a predetermined range, and adjusting the pH of the liquid.

18. A method according to claim 17, wherein adjusting the pH of the liquid includes adding salts or minerals to the liquid.

19. A closed hydraulic system comprising:
a first vessel, wherein the first vessel is a vessel according to claim 1;
a pump;
a liquid tank;
a heat exchanger;
a first pipe between the liquid tank and the vessel;
a second pipe between the vessel and the heat exchanger; and
a third pipe between the heat exchanger and the liquid tank,
wherein the pump is capable of continuous circulating flow of liquid through the vessel via the first, second and third pipe under hydraulic pressure.

20. A closed hydraulic system of claim 19 further comprising a second vessel, wherein the second vessel is a vessel according to claim 1.

21. A closed hydraulic system of claim 20, wherein the first and second vessels are used to perform the same process from among a mashing process, a sparging process, a boiling process and a fermentation process.

22. A closed hydraulic system of claim 20, wherein the first and second vessels are used to perform a different process from among a mashing process, a sparging process, a boiling process and a fermentation process.
